(19) United States
(12) Patent Application Publication
(10) Pub. No.: US 2025/0121063 A1
(43) Pub. Date: Apr. 17, 2025

(54) COMPOSITIONS OF LEUKOCYTES WITH RESISTANCE TO PROSTANOIDS AND RELATED METHODS OF USE

(71) Applicant: Van Andel Research Institute, Grand Rapids, MI (US)

(72) Inventors: Russell Jones, Grand Rapids, MI (US); Michael Dahabieh, Grand Rapids, MI (US)

(21) Appl. No.: 18/913,303

(22) Filed: Oct. 11, 2024

Related U.S. Application Data
(60) Provisional application No. 63/589,902, filed on Oct. 12, 2023.

(51) Int. Cl. A61K 39/00; A61P 35/00; C07K 14/705; C12N 5/0783; C12N 15/113

(52) U.S. Cl. CPC A61K 40/34; A61K 40/11; A61P 35/00; C07K 14/705; C12N 5/0636; C12N 15/1138; A61K 40/50; C12N 2310/11; C12N 2310/531

(57) ABSTRACT

A composition includes genetically modified leukocytes, the modification including a deleted or silenced prostacyclin receptor PTGIR that confers resistance to the immunosuppressive effects of prostanoids.

### CLAIM FOR PRIORITY

This application claims priority under 35 U.S.C. § 119 (e) to U.S. Patent Application No. 63/589,902 filed on Oct. 12, 2023, which is hereby incorporated by reference in its entirety.

### REFERENCE TO THE SEQUENCE LISTING

The instant application contains a Sequence Listing which has been submitted electronically in XML format and is hereby incorporated by reference in its entirety. Said XML copy, created on Oct. 10, 2024, is named 232119-555234_SL and is 33,473 bytes in size.

### FIELD OF THE INVENTION

This invention relates to adoptive cellular therapy, and more specifically to genetic modification of the prostacyclin receptor (PTGIR) gene in immune cells.

### BACKGROUND

Chemotherapy is the standard of care to treat many types of cancer. When chemotherapy is or becomes ineffective, alternative methods for treating cancer are needed. For example, systems, mechanisms, and components of the human immune system sometimes can prevent or slow the growth of cancerous cells through recognition by T cells. In order to improve the efficacy of immune cells to reduce the number of or kill cancer cells, T cells can be isolated from the blood of a patient and genetically modified or altered. When administered back into the patient, the modified cells more efficiently act on the cancer cells as anti-tumor immunotherapy.

In anti-tumor immunotherapy, certain tumors may contain cells that suppress or regulate anti-tumor cells, thereby reducing the effectiveness of an anti-tumor response. In adoptive immunotherapy, transferred cells may produce undesirable side effects. Other approaches to controlling undesired adoptive cell function is to include have used cell surface markers or suicide genes that allow the selective destruction of the gene modified cells.

Therefore, it is an objective of the present invention to provide genetically modified leukocytes, a method of genetically modifying leukocytes, and a method of using these leukocytes, including in the treatment of cancer.

### SUMMARY

This section provides a general summary of the disclosure and is not a comprehensive disclosure of its full scope or all its features.

This disclosure relates to the genetic modification of the PTGIR gene in immune cells.

In certain embodiments, the modified immune cells may be used in adoptive T cell therapies to enhance immune responses against cancer.

In general, a population of leukocytes comprises CD8 T-cells that have been genetically modified to reduce or silence expression in the CD8 T-cells of a functional prostaglandin 12 receptor (PTGIR) gene or of a functional PTGIR protein. In some embodiments, reduced expression of a functional PTGIR includes decreasing the expression of a functional protein or expressing a non-functional protein.

In some embodiments, the expression of PTGIR is disrupted by introducing an interfering RNA or trigger RNA into the CD8 T-cells.

In some embodiments, the interfering RNA is a short hairpin RNA (shRNA).

In some embodiments, the interfering RNA is small interfering RNA (siRNA).

In some embodiments, the expression of PTGIR is disrupted with a CRISPR system.

In some embodiments, the expression of PTGIR is disrupted by knocking down the PTGIR.

In some embodiments, the expression of PTGIR is disrupted by knocking out the PTGIR.

In some embodiments, the expression of PTGIR is disrupted by the prevention of ribosomes from synthesizing the PTGIR.

In some embodiments, the expression of PTGIR is disrupted by marking the mRNA that codes for PTGIR for destruction.

In some embodiments, the disrupted expression of PTGIR improves anti-tumor T-cell responses.

In some embodiments, the disrupted expression of PTGIR enhances antigen-specific T-cell expansion in chronic infection.

In some embodiments, the disrupted expression of PTGIR increases levels of Wt P14 IFNγ+/TNFα+ cells.

In some embodiments, the CD8 T-cells are regulatory T-cells (Tregs).

In some embodiments, the CD8 T-cells are suppressor T-cells.

In some embodiments, the CD8 T-cells are tumor infiltrating lymphocytes.

In general, a pharmaceutical composition comprises a population of genetically modified CD8 T-cells having a knocked out PTGIR, and a pharmaceutically acceptable carrier or diluent.

In some embodiments, wherein the leukocytes include an expression vector

In general, a method of disrupting expression of prostaglandin 12 receptor (PTGIR) in a CD8 T-cell comprises introducing an interfering RNA into a CD8 T-cell, allowing the interfering RNA to bind an mRNA that codes for PTGIR, reducing or silencing the expression of PTGIR, reducing prostacyclin binding, and enhancing CD8 T cell activity.

In general, a method of treating cancer comprises collecting CD8 T-cells from a subject.

isolating the collected CD8 T-cells, genetically modifying the isolated CD8 T-cells such that the expression of a prostaglandin 12 receptor (PTGIR) in the CD8 T-cells is disrupted by reducing the PTGIR or preventing the Ptgir gene in the CD8 T-cells from expressing a functional protein and administering a pharmaceutically effective amount of the modified isolated CD8 T-cells into a patient. In some embodiments, the subject from who the CD8 T-cells were collected, and the patient are the same. In some embodiments, the subject from who the CD8 T-cells were collected, and the patient are different.

Other features and advantages of the invention will become apparent from the following detailed description, taken in conjunction with the accompanying drawings, which illustrate, by way of example, various features of embodiments of the invention.

### BRIEF DESCRIPTION OF DRAWINGS

The drawings described herein are for illustrative purposes only, are intended to be restrictive or to limit the scope of the present disclosure. Exemplary embodiments are illustrated in referenced figures.

FIG. 1 shows the silencing of PTGIR improves anti-tumor T cell responses in mouse T cells.

FIG. 2 shows that mice that received the shPTGIR OT-1 cells had increased survival.

FIG. 3 shows transduction of shPTGIR into effector T-cells results in a robust immune response and T-cells that have reduced phenotypic markers of exhaustion.

FIG. 4 shows T-cells carrying the CD45.2 surface marker, T-cells transduced with shPtgir had higher absolute abundance (right bar charts) and lower percentage of Tim-3 (left bar chart).

FIG. 5 shows intracellular staining for interferon gamma (IFNγ) and tumor necrosis factor alpha (TNFα), showing that these shPTGIR T-cells recovered from the animals are capable of responding to antigen stimulation, which is further evidence supporting their non-exhausted state.

FIG. 6 shows PTGIR expression is correlated with an elevated NRF2 gene signature.

FIG. 7A shows relative PTGIR protein expression (normalized to ACTN4) in WT OT-I T cells following transduction with empty vector (CTRL) or PTGIR-expressing (PTGIR OB) retroviral vector (mean+/− SEM, n=3)

FIG. 7B shows B16-OVA melanoma tumor growth in mice that received adoptive transfer of control (CTRL) or PTGIR-expressing (PTGIR OE) WT OT-I cells at 7 dpti (mean+/−SEM.

FIG. 8 shows probability of survival as a function of time for human cancer patients bearing CD8+ T-Cells with low PTGIR expression.

### DETAILED DESCRIPTION OF THE INVENTION

CD8+ T cells seek and destroy abnormal host cells. Immune checkpoints (IC) are inhibitory receptors expressed on immune cells that trigger immunosuppressive signaling pathways in order to minimize collateral damage. Signaling through these molecules can drive immune cells (i.e. CD8+ T cells) into a state of exhaustion-defined by: reduced effector function; sustained expression of IC molecules; reduced CD8+ T cell abundance; triggering death of CD8+ T cells; and distinct transcriptional and metabolic state versus effector or memory cells. A deeper understanding of molecular signatures of exhausted CD8+ T cells can illuminate alternative molecular interventions to “reverse” exhaustion.

### Nuclear Factor Erythroid 2 (NRF2) Pathway

The NRF2 Pathway is well characterized. However, very little has been studied regarding NRF2 Pathway in CD8+ T cells. It is known that Kelch-like ECH-associated protein 1 (KEAP1) is the negative regulator of NRF2 (Vos), and that KEAP1″ T cell model mimics a state of high oxidative stress (DeCamp). The loss of KEAP1 was also shown to promote NRF2 activity in CD8+ T cells (Sheldon et al). However, the prostacyclin receptor (PTGIR) has never been characterized in CD8+ T cells.

Applicants have studied the NRF2 activity and PTGIR expression in CD8+ T cells. Applicants found that Ptgir mRNA is highly expressed in terminally exhausted CD8+ tumor infiltrating lymphocytes (TILs) from mice and is correlated with an elevated NRF2 gene signature. This is described in more detail in Example 7.

### Modulation of PTGIR in CD8+ T Cells

Applicants studied the modulation of PTGIR in CD8+ T cells and discovered that the prostacyclin receptor represents a novel immune checkpoint that is not regulated by a protein-protein interaction, but rather by a protein-lipid derived metabolite (prostacyclin) interaction. Targeting PTGIR could be an alternative means to override the cytoprotective effects of the NRF2 pathway and boost CD8+ T cell function.

Phospholipase A2 liberates arachidonic acid (AA) from membrane phospholipids. COX-1/COX-2 initializes the conversion of AA to prostaglandin (PGH2). PGH2 is converted to PGI2 via prostacyclin synthase. When PGI2 binds to the prostacyclin receptor, adenylate cyclase (Ac) converts ATP to CAMP and engages the PKA signaling pathway.

In general, if adoptively transferred leukocytes had resistance to prostanoids but suppressor or regulatory cells within a tumor remained responsive to the immunosuppressive effects of glucocorticoids, the administration of prostanoid resistant leukocytes to a patient would preserve the effector activity of the adoptively transferred gene modified leukocytes. To make these anti-tumor leukocytes, during their ex vivo culture the cell population can be transduced or transected or co-transduced or co-transfected with vectors containing a gene for antisense PTGIR or subject to genetic modifications that reduce or abolish the function of the PTGIR gene, e.g a CRISPR-mediated mutation that destroys functional expression of PTGIR gene product at one or more PTGIR alleles. A preferred embodiment is where at least ten percent of genetically modified leukocytes express a gene that confers resistance to prostanoids.

### Prostaglandin 12 (Aka Prostacyclin)

Conventionally, it was believed that Prostaglandin 12 (aka prostacyclin) acts in a “positive” manner to promote (license/permit) the suppressive function of Tregs to have control over other immune cells. This ‘intended’ suppressive activity of Tregs—stimulated by PGI1 ligand through receptor PTGIR-reduced deleterious asthma type activity in an animal model of airway allergy. It was believed that knocking out PTGIR caused worse allergy, as human mutants with degraded PTGIR activity correlated with allergy (asthma). It was believed that prostacyclin signaling licenses Treg suppressive function and prevents reprogramming toward a pathogenic Treg phenotype. PGI2 has been FDA approved to treat pulmonary hypertension for 3 decades and it was believed that a PGI2 analog could be repurposed to increase the ability of Treg to suppress allergic inflammation or other inflammatory diseases, such as autoimmunity.

Applicants discovered that prostacyclin has a negative effect on T cell therapy, and that blocking its effect improved the function of the T cell therapy.

### Definitions

Unless otherwise defined, all terms of art, notations and other scientific terms or terminology used herein are intended to have the meanings commonly understood by those of skill in the art to which this disclosure pertains. In some cases, terms with commonly understood meanings are defined herein for clarity and/or for ready reference and understanding, and the inclusion of such definitions herein should not necessarily be construed to mean a substantial difference over what is generally understood in the art. One skilled in the art will recognize many methods and materials similar or equivalent to those described herein, which could be used in the practice of the present invention. Indeed, the present invention is in no way limited to the methods and materials described. For purposes of the present invention, the following terms are defined below.

Articles such as “a,” “an,” and “the” may mean one or more than one unless indicated to the contrary or otherwise evident from the context. The term “and/or” means one or all of the listed elements or a combination of any two or more of the listed elements.

Embodiments of the present disclosure will employ, unless otherwise indicated, techniques of medicine, organic chemistry, biochemistry, molecular biology, pharmacology, and the like, which are within the skill of the art. Such techniques are explained fully in the literature. As appropriate, procedures involving the use of commercially available kits and/or reagents are generally carried out in accordance with manufacturer's guidance and/or protocols and/or parameters unless otherwise noted.

In the following description, numerous specific details are given to provide a thorough understanding of the embodiments. The embodiments can be practiced without one or more of the specific details, or with other methods, components, materials, etc. In other instances, well-known structures, materials, or operations are not shown or described in detail to avoid obscuring aspects of the embodiments.

Reference throughout this specification to “one embodiment,” “an embodiment,” or “embodiments” means that a particular feature, structure, or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the invention.

Unless indicated otherwise, when a range of any type is disclosed or claimed, it is intended to disclose or claim individually each possible number that such a range could reasonably encompass, including any sub-ranges encompassed therein. Moreover, when a range of values is disclosed or claimed, which Applicant intends to reflect individually each possible number that such a range could reasonably encompass, Applicant also intends for the disclosure of a range to reflect, and be interchangeable with, disclosing any and all sub-ranges and combinations of sub-ranges encompassed therein.

2A self-cleaving peptides, or 2A peptides, is a class of 18-22 aa-long peptides, which can induce the cleaving of the recombinant protein in cell. 2A peptides are derived from the 2A region in the genome of virus. The members of 2A peptides are named after the virus in which they have been first described. For example, F2A, the first described 2A peptide, is derived from foot-and-mouth disease virus. Exemplary 2A peptides include, but are not limited to, P2A, E2A, F2A and T2A, with the following sequences (adding the sequence “GSG” (Gly-Ser-Gly) on the N-terminal of a 2A peptide is optional).

“Adoptive cellular therapy,” “adoptive cell therapy,” or “adoptive cell transfer” (ACT) refers to the treatment of a disease by the adoptive transfer of hematologic cells including leukocytes to a patient whereby the hematologic cells modulate a disease and/or its symptoms. Adoptive cellular therapy includes, but is not limited to, the use of: blood or platelet transfusions; donor-derived anti-viral lymphocytes to treat viral infections; tumor infiltrating lymphocytes (TILs) for cancer treatment; chimeric antigen receptor bearing T-cells (CAR-T) for cancer; lymphocytes that have been selected for, or genetically modified to drive, expression of anti-tumor T-cell receptor genes; natural killer cells for cancer treatment; antigen presenting cells such as dendritic cells or macrophages that present microbial, viral or tumor antigens; hematopoietic stem cell transplantation whereby hematopoietic progenitors are delivered, often contained within populations of bone marrow, peripheral blood (with or without mobilization of hematopoietic precursors), umbilical cord blood or enriched precursor cells (e.g., CD34+ cells); hematopoietic cell grafts with and populations of leukocytes for use in graft-versus-leukemia or graft-versus-tumor responses; transfusions of leukocytes or their precursors to treat acquired or congenital leukopenias and immune deficiencies; leukocytes including CD3+ T-cells to promote immune reconstitution following hematopoietic ablation and hematologic stem cell transplantation. Cells used in ACT may be obtained or derived from the recipient of the ACT (i.e., self or autologous cell population), from another individual or individuals (“non-self”), or some mixture of self and non-self. Cells used in ACT may be genetically modified.

“Adoptive cellular immunotherapy” or “adoptive cell immunotherapy” refers to a type of adoptive cellular therapy where an immune cell is delivered into a mammal to affect a beneficial result. Examples of adoptive cellular immunotherapy include, but are not limited to, anti-viral T cells, CAR-T cells, tumor infiltrating lymphocytes (TILs) and natural killer cells.

“Auto-immune disease,” “auto-immune disorders,” or “auto-immunity” refers to or describes a condition in mammals where an immune response interferes with, or causes damage to normal cells, tissues or physiological processes. Examples of auto-immune disorders include but are not limited to alopecia areata, antiphospholipid antibody syndrome (aPL), autoimmune hepatitis, Celiac disease, diabetes type 1, eosinophilic esophagitis, Graves' disease, Guillain-Barre syndrome, Hashimoto's thyroiditis, hemolytic anemia, Idiopathic thrombocytopenia purpura (ITP), Inflammatory bowel disease (IBD), ulcerative colitis, inflammatory myopathies, multiple sclerosis, myasthenia gravis, primary biliary cirrhosis, Rheumatoid arthritis (adult and juvenile), scleroderma, Sjögren's syndrome, Systemic lupus erythematosus (SLE), vitiligo.

“b,” “B,” “beta” and “0” when used as prefixes in definitions of molecules are used equivalently and interchangeably.

“Beneficial results” as used herein may include, but are not limited to, lessening or alleviating the severity of the disease condition, preventing the disease condition from worsening, curing the disease condition, preventing the disease condition from developing, lowering the chances of a patient developing the disease condition, prolonging a patient's life or life expectancy and reducing side-effects.

“Cancer” and “cancerous” refer to or describe a condition in mammals that is typically characterized by unregulated cell growth. Examples of cancer include, but are not limited to carcinomas, sarcomas, B-cell lymphomas (Hodgkin's lymphomas and/or non-Hodgkins lymphomas, Burkitts' lymphoma), leukemias, T-cell lymphomas, multiple myelomas, brain tumor, breast cancer, histiocytosis, colon cancer, lung cancer, hepatocellular cancer, gastric cancer, pancreatic cancer, cervical cancer, ovarian cancer, liver cancer, bladder cancer, cancer of the urinary tract, thyroid cancer, renal cancer, carcinoma, melanoma, nasopharyngeal cancer, head and neck cancer, brain cancer, and prostate cancer, including but not limited to androgen-dependent prostate cancer and androgen-independent prostate cancer.

“Co-express” refers to simultaneous expression of two or more genes. Genes may be nucleic acids encoding, for example, a single protein or a chimeric protein as a single polypeptide chain. In an embodiment, the first and second polynucleotide chains are linked by a nucleic acid sequence that encodes a linker polypeptide that is capable of being cleaved. In another embodiment, the first and second polynucleotide chains are driven by independent promoters. In another embodiment the polynucleotides may be linked by internal ribosome entry sequence (IRES) or a functionally equivalent sequence. Alternately, the genes are encoded by two different polynucleotides and are instead present on, for example, two different vectors. If the aforementioned sequences are encoded by separate vectors, these vectors may be simultaneously or sequentially transfected or transduced.

“Conditions,” “disease conditions,” “diseases” and “disease state” include physiological states in which a disease or symptom is manifest. Examples of conditions include cancer, infection, auto-immunity, graft failure and delayed hematopoietic reconstitution.

“Effector function” refers to the specialized function of a cell. For example, an effector function of a T-cell may be cytolytic activity, helper activity, suppressor activity, regulatory activity and may include the secretion of cytokines when the cell is stimulated. Effector function may act locally to the cell (e.g. cytolytic activity), distally (e.g. secretion of cytokines) or both locally and distally (e.g. secretion of cytokines).

“Electroporation” refers to the administration of an electric current to a cell or population of cells so that nucleic acid present outside the cell is rapidly brought into the cell. Electroporation is a form of transfection.

“Exhaustion” refers to an immune cell's state of reduced ability to function, including effector function, sustained expression of immune checkpoint molecules, reduced CD8+ T cell abundance, and excessive apoptosis (cell death).

“Express” or “expression” refers to the production of a protein either directly from a gene under steady-state conditions or production as a result of induction of expression of that gene by a factor from outside the cell.

“Insert,” “payload” or “gene” refers to a polynucleotide that is delivered into a cell to cause genetic modification. These phrases may also define genes, “foreign” genes or sequences and “transgenes.” Gene refers to polynucleotide that is introduced into a cell, or may refer to a polynucleotide or a site of targeting within a cell. When referring to a site of chromosomal genetic modification, a particular genomic location may be referred to terms including but not limited to a gene, a locus, an allele and may be identified by position on chromosomal maps. Often, but not required, a gene may be capable of producing an RNA transcript or being recognized by DNA or RNA processing machinery (for example a payload comprising an exogenous gene promoter that would be inserted in front of the coding region of a given gene in order to drive gene expression in a situation where otherwise the gene's endogenous promoter would not drive expression). Often, but not required, the payload is delivered as part of a vector. Payload may include regulatory or control sequences, such as start, stop, promoter, signal, disruptive sequences (e.g. sites for homologous recombination), anti-sense sequences, RNA stability or RNA regulation sequences, internal ribosome entry sequences, signal for protein secretion or targeting to organelles, or other sequences used by a cell's genetic, transcriptional and translational machinery. The gene or sequence may include nonfunctional sequences or sequences with no known function. A host cell that receives introduced DNA or RNA has been “transformed” and is a “transformant.” The invention also contemplates DNA sequences that encode the same desired protein by alternative codon usage.

Gene name abbreviations used herein (e.g. PTGIR), unless otherwise specified, refer to the human gene. The corresponding genes, names and gene name abbreviations for other species are readily obtained by one skilled in the art to which this invention belongs. To the extent the meaning from the context is recognized by one of ordinary skill in the art, no distinction is made in the nomenclature between the human gene and the human gene product (protein) in the application, as we have not adhered to the HUGO Gene Nomenclature Committee of italicized text for human gene symbols and non-italicized for the human protein.

“Genetically modified cells”, “gene modified cells”, “redirected cells” or “genetically engineered cells” refer to cells or cell types that have had their complement of DNA or RNA altered by external action. Many methods for such modification are known and include but are not limited to transduction of cells using a viral or viral-based vector, transfection of an expression vector (often a plasmid), introduction of enzymes or enzyme systems with additional components whereby those systems modify a cell's DNA and or RNA complement. Such systems include Transcription activator-like effector nucleases (TALENS), Zinc finger proteins and clustered regularly interspaced short palindromic repeats (CRISPR)/Cas9. Other systems genetic modifications approaches use transposons in systems such as Sleeping Beauty. For example, one type of “genetically modified leukocyte” is a leukocyte that contains a gene that confers PTGIR resistance, and which also co-expresses a transgene encoding for a recombinant T-cell receptor gene or a chimeric T cell antigen receptor. Genetically modified cells may be created by the action of man (e.g. delivery of a protein of, or RNA encoding for, a recombinase or integrase enzyme), by nature (e.g. by a viral infection such as Epstein Barr Virus or a wart virus) or a combination of both man and nature. Genetic modifications also include but are not limited to modifications to the cell's structures around DNA and RNA which includes epigenetic modifications of DNA, RNA (e.g. methylation of nucleotide bases) and post-translational modification of proteins involved in the regulation of the function of DNA and e.g. acetylation or methylation of chromosomal histones.

“Immune cell” refers to a leukocyte which has a direct role in an immune response or which has immune cell function.

“Immune cell function” refers to a cell's known or potential function in an “immune response” and may or may not include other activities which may include, but are not limited to, removal of cellular and tissue debris including enucleation of erythrocytes, maturation of erythroid cells, maturation of platelets, production of cytokines and pro-inflammatory factors, promoting apoptosis or anergy in leukocytes, providing survival signals to leukocytes, immune surveillance, migration, antigen presentation, maintaining an immune system, anti-viral cytotoxicity, anti-cancer cytotoxicity, promoting engraftment of transplanted hematopoietic stem and progenitor cells, anti-helminth activity, phagocytosis of microbes. wound repair, bone repair, promoting immune tolerance and antibody-dependent cell mediated cytotoxicity (ADCC). Immune cell function, like aspects of immune response, may promote the health of a mammal or may be deleterious, for example, by causing autoimmunity or graft rejection.

“Immune response” refers to immune activities including, but not limited to: innate immunity, humoral immunity, cellular immunity, immunity, inflammatory response, acquired (adaptive) immunity, autoimmunity and/or overactive immunity, breaking of immune tolerance, graft rejection, response to allo- and xeno-antigens, graft-versus-leukemia activity, graft-versus-tumor activity, graft-versus-host disease, promoting immune tolerance and includes immune responses produced by adoptive cellular therapies.

“Leukocyte” refers to a cell of the blood cell lineage. Leukocytes include, but are not limited to, alveolar macrophages, antigen presenting cells, B-cells, basophils, cytotoxic T-cells, dendritic cells, epithelioid cells, eosinophils, giant cells, granulocytes, helper T-cells, histiocytes, Kupffer cells, Langerhans cells, large granular lymphocytes, leukocyte precursors, lymphocytes, macrophages, mast cells, memory cells, microglia, monocytes, monoosteophils, myeloid dendritic cells, natural killer cells, natural killer T cells, neutrophils, osteoclasts, phagocytes, plasma cells, plasmacytoid dendritic cells, regulatory T-cells (Tregs), suppressor T-cells, T-cells and tumor infiltrating basophils. Leukocytes are distinguishable from two other lineages of the blood cells-erythroid cells (wherein maturing erythrocytes contain substantial levels of hemoglobin protein) and megakaryocytes and platelets. Leukocyte as used herein refers to a non-erythroid, non-megakaryocytic hematologic cell regardless of whether the leukocyte has been derived from a normal physiological hematopoietic process of a mammal, e.g. is a cell of, or is a cell derived from, a sample obtained from a human patient or donor, or whether the leukocyte was generated from an alternate population of cells, such as, and not limited to, a leukocyte generated in vitro from precursors or progenitors derived from other sources of cells including, but not limited to, embryonic stem cells (ESC) or induced pluripotent stem cells (iPSC). The forgoing definitions are not limiting—other cell populations with lesser potency (multi-potent, bi-potent or uni-potent) may be used as sources of leukocyte precursors or progenitors instead of pluripotent or totipotent cells. The forgoing cell populations cells may be transformed. Mature and maturing leukocytes may be found throughout the human body including without limitation, circulating in the peripheral blood (e.g. neutrophils, eosinophils, basophils, T-cells, B-cells, macrophage/monocytes, dendritic cells), in the lymphatics, the spleen, the liver, in the primary and secondary lymphoid organs and in the central nervous system. Leukocytes may be resident in tissues (e.g. microglia in the central nervous system, tissue macrophages or osteoclasts in bone tissue). Some leukocytes migrate and traffic through tissues and organs and adopt new phenotypes depending on their history, function and location, by way of example monoosteophils being produced from the monocytes and macrophage lineage. Macrophages may fuse to give rise to giant cells. Although the anatomical location of a leukocyte may give clues to the type of leukocyte or its function, classification of a leukocyte requires a multi-parametric analysis based on definitions in the field for each leukocyte sub-population that are in use at the time of analysis.

Sub-populations of leukocytes are generally defined by parameters such as cell size, shape, intracellular granularity and degree of surface regularity, cell surface markers, gene expression including specialized genes such as the T-cell receptor, immunoglobulin heavy and light chains, and cellular function. These parameters can be examined by many means known in the field including, but not limited to, electromagnetic radiation including optically by cell analyzers, light microscopy with and without histological stains, the use of antibodies, aptamers and with means of detection (e.g. fluorochromes, quantum dots, enzyme staining) in combination with techniques such as cell imaging, flow cytometry or mass-spectroscopy-cytometry, analysis of intracellular markers by assays including granule types and contents, enzyme function, permeabilization for antibody or aptamer staining of intracellular antigens including cytokines, functional studies (e.g., phagocytosis, motility and chemotaxis, degranulation, capacity to undergo mitosis in response to cytokines or in response to stimuli such as aggregation of cell surface antigens by cross-linking antibodies or by mitogens, ability to stimulate, suppress or attract other leukocytes, ability to kill target cells or kill or ingest pathogens, ability to degrade, remodel or form bone), gene expression analysis, impedance analysis and cell adhesion noise (CAN-Q), adherence to substrates including plastic or antibody coated beads or columns. Leukocyte precursors can be defined using the same parameters as listed herein, plus additional studies that may be performed to evaluate the potency of such precursors-including which cell types can be produced from the precursor cell and the precursor's proliferation potential-using in vitro or in vivo studies. T-cells and B-cells are examples of leukocytes where sub-populations of these cells continue to be identified. Leukocytes that have undergone ex-vivo manipulation may display different phenotypes when compared to the original cells or other leukocytes. This phenotypic difference may be particularly evident when leukocytes are maintained in culture for hours, days or weeks, including under culture conditions that drive mitosis.

In another aspect, leukocyte as used herein also refers to a cell obtained from a leukocytic leukemia, lymphoma, histiocytosis or dysplasia. In yet another aspect, leukocyte as used herein also refers to a cell of a cell line, regardless of whether that cell line is stable or unstable, transformed or untransformed, where that cell line is derived from leukocytes, leukocyte precursors or leukocyte progenitors. Examples of leukocyte cell lines used for human clinical studies include “GRm13Z40-2”, a cytotoxic T-lymphocyte cell line genetically modified by the targeted disruption of GR alleles and Neukoplast (NK-92), a natural killer cell line.

In various aspects, the definition of leukocyte and classification of leukocyte type also anticipates that some mammalian leukocytes and the precursors of some hematologic lineages may display plasticity, i.e. an ability to develop and differentiate, or de-differentiate, between two or more lineages that were otherwise believed to be distinct paths of development and maturation, e.g., see “Transdifferentiation of Malignant B-Cells into Macrophages in a Murine Model of Burkitt's Lymphoma”, Bruns et al. (2014) Blood, vol. 124 no. 21 5406 and references therein.

“Mammal” refers to any member of the class Mammalia, including, without limitation, humans and nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be included within the scope of this term.

“Patient” as used herein refers to a mammal who is pursing or in need of treatment.

“Polynucleotide” includes but is not limited to DNA, RNA, cDNA (complementary DNA), mRNA (messenger RNA), rRNA (ribosomal RNA), shRNA (small hairpin or short hairpin RNA), snRNA (small nuclear RNA), snoRNA (short nucleolar RNA), miRNA (microRNA), anti-mirs (also known as antagomirs), small activating RNAs (saRNAs), genomic DNA, synthetic DNA, synthetic RNA, and/or tRNA and hybrids between a strand of DNA and a strand RNA form.

“Prostanoids” are the parent group of prostacyclins and prostaglandins, and the receptor, PTGIR binds to both (in different degrees).

“Resistance” refers to reducing the effectiveness of a protein, drug, compound or other condition acting on a cell, whereby a cell which has resistance (“resistant cell”) shows a lower responsiveness to the protein, drug, compound or condition when compared to a non-resistant cell. Resistance may be overcome or reversed by reducing, eliminating or defeating the mechanisms which underpin resistance.

“Responsiveness” refers to the measurable response of a cell. Techniques used to measure responsiveness include but are not limited to: performing laboratory analyses and assays including but not limited to measuring the number of cells, the persistence of cells, the growth of cells, the survival of cells, evaluating the cell's ability to migrate; evaluating the cell's ability to interact with target cells and effects on growth, gene expression, cytokine production, phenotype, report gene activity and cell function. Responsiveness can also be evaluated in vivo by, for example, clinical laboratory measurements and observing clinical outcomes.

A “subject” as used herein refers to a mammal.

“Target cell” refers to a cell that is the target of a treatment, immune response or immune cell function. Without limiting the forgoing, by way of some examples, a target cell may be a cancer cell (the target of a cytolytic T cell), a T cell (the target of a suppressor T cell), a T cell (the target of an antigen presenting cell), a B cell (the target of a T helper cell), a transformed epithelial cell i.e. a wart cell (the target of ointment containing anti-wart compound).

The terms “T-cell” and “T-lymphocyte” are interchangeable and used synonymously herein. Without limiting the forgoing, by way of some examples include but are not limited to naive T cells, central memory T cells, effector memory T cells, memory T cells, regulatory T cells, suppressor T cells or combinations thereof.

The terms “transduction” and “transfection” are defined separately herein but share a common basis in delivering a polynucleotide into a cell and are often used synonymously herein. We refer to transfection as a form of polynucleotide delivery that utilizes viruses, viral vectors or components of viruses. “Transduction” refers to the introduction of an exogenous polynucleotide into a cell using a viral vector or components of viruses. “Transfection” refers to the introduction of a exogenous polynucleotide into a cell using a non-viral means. The term “transformation” means the introduction of a polynucleotide comprising a DNA or RNA sequence to a host cell. Transformation may result in the host cell replicating the DNA or RNA sequence, or may result in expression of the introduced DNA or RNA sequence to produce a desired substance, such as a protein or enzyme coded by the introduced DNA or RNA sequence or may simply result in the action of DNA or RNA-without replication or expression-on the DNA or RNA complement of the cell. An example of the latter is the delivery of anti-sense and RNA interference oligonucleotides. The term “transformant” means the cell which has been transformed. A transformant may be a microbe or animal cell. The polynucleotide e.g., DNA or RNA introduced to a host cell can come from any source, including cells of the same genus or species as the host cell, or cells of a different genus or species. A physical process or chemical agent may be applied to assist with the delivery of a polynucleotide. Such physical processes include electroporation and such chemical agents include liposomes and chemical agents that associate with polynucleotides to promote for uptake into cells by endocytosis (including receptor mediated endocytosis), phagocytosis, pinocytosis, emperipolesis and vesicle fusion.

In various aspects, “transformed” has two meanings. One related to transfection, above, the second meaning defined below. In combination, this can lead to sentences wherein both definitions are in use, by way of example and with explanations in square brackets: “we can transfect a cell with a vector and transfection agent wherein that vector contains a transforming oncogene [useful to generate a transformed cell], leading to a transformant [cell post-gene delivery] and resultant transformation [process] of the target cell [into a transformed cell]”. “Transformed” as used herein has a second meaning with respect to cells whereby a transformed cell is a cell that has undergone a genetic or phenotypic change to permit sustained growth in tissue culture or a system of animal passage, where such a transformed cell may display one of more properties of: tumor formation with or without spread, growth factor independence, colony formation, ability to undergo serial passaging in culture and loss of contact inhibition. The process by which a cell is transformed here is “transformation”. One form of transformation is malignant transformation. A transformed cell is often associated with genetic changes, and such changes may be induced by an external action (e.g. by a chemical, physical (e.g., alpha particle) or energetic (e.g. X-ray, gamma ray) mutagen, by a virus or vector containing one or more genes capable of promoting transformation, by fusion with an already transformed cell), by spontaneous genetic re-arrangements or mutations within a cell to result in a transformed phenotype, or by a combination of external action and spontaneous genetic re-arrangements or mutations within a cell.

“Treatment” and “treating” refer to both therapeutic treatment and prophylactic or preventative measures, wherein the object is to prevent or slow down (lessen) the targeted pathologic condition, prevent the pathologic condition, pursue or obtain beneficial results, or lower the chances of the individual developing the condition even if the treatment is ultimately unsuccessful. Those in need of treatment include those already with the condition as well as those prone to have the condition or those in whom the condition is to be prevented. Treatment also includes medical intervention to reduce side effects of a previous or concurrent treatment, or the selection of a treatment regimen that is expected to minimize side effects.

“Tumor” refers to all neoplastic cell growth and proliferation, whether malignant or benign, and all pre-cancerous and cancerous cells and tissues.

“Vector,” “cloning vector” and “expression vector” as used herein refer to the vehicle by which a polynucleotide sequence (e.g. a foreign gene) can be introduced into a host cell, so as to transform the host and promote expression (e.g. transcription and translation) of the introduced sequence. Vectors include plasmids, phages, viruses, etc. Viral vectors which may be used include but are not limited to lentiviral vectors, retroviral vectors, foamy virus vectors, adeno-associated virus (AAV) vectors, hybrid vectors and/or plasmid transposons (for example sleeping beauty transposon system) or integrase-based vector systems. Other vectors that may be used in connection with alternate embodiments of the invention will be apparent to those of skill in the art.

### Control Elements

Appropriate transcriptional/translational control signals and protein coding sequences are described in, for example, Sambrook et al., Molecular Cloning: A Laboratory Manual, 3d Ed. (Cold Spring Harbor Laboratory 2001). These techniques may include in vitro recombinant DNA and synthetic techniques and in vivo recombination, e.g., in vivo homologous recombination. Expression of a nucleic acid sequence may be regulated by a second nucleic acid sequence that is operably linked to the polypeptide-encoding sequence.

Exemplary expression control elements useful for regulating the expression of an operably linked coding sequence include, but are not limited to, inducible promoters, constitutive promoters, secretion signals, and other regulatory elements. When an inducible promoter is used, it can be controlled, e.g., by a change in nutrient status, or a change in temperature, in the host cell medium.

### Expression Vectors

Expression vectors capable of being replicated in a bacterial or eukaryotic host comprising a nucleic acid encoding a polypeptide (or protein) are used to transfect a host and thereby direct expression of such nucleic acid (e.g., genes that confer resistance to glucocorticoid) to produce the polypeptide (or protein). Exemplary mammalian expression vectors contain both prokaryotic sequences, to facilitate the propagation of the vector in bacteria, and one or more eukaryotic transcription units that are expressed in eukaryotic cells. The pcDNAI/amp, pcDNAI/neo, pRc/CMV, pSV2gpt, pSV2neo, pSV2-dhfr, pTk2, pRSVneo, pMSG, pSVT7, pko-neo and pHyg derived vectors are examples of mammalian expression vectors suitable for transfection of eukaryotic cells. Transfection methods may include chemical means, e.g., calcium phosphate, DEAE-dextran, or liposome; or physical means, e.g., microinjection or electroporation. In some embodiments, electroporation is used for transfecting leukocytes with an expression vector containing the insert of antisense against PTGIR.

The transfected cells are grown up by techniques such as those described in Kuchler et al. (1977) Biochemical Methods in Cell Culture and Virology. In various embodiments, the host cell line is mammalian origin, and particularly, human origin.

Numerous expression vector systems may be employed. For example, one class of vector utilizes DNA elements which are derived from animal viruses such as bovine papilloma virus, polyoma virus, adenovirus, adeno-associated virus, herpes simplex virus-1, vaccinia virus, baculovirus, retroviruses (RSV, MMTV or MOMLV) or SV40 virus. Others involve the use of polycistronic systems with internal ribosome binding sites. Additionally, cells which have integrated the DNA into their chromosomes may be selected by introducing one or more markers which allow selection of transfected host cells. The marker may provide for prototrophy to an auxotrophic host, biocide resistance (e.g., antibiotics) or resistance to heavy metals such as copper. The selectable marker gene can either be directly linked to the DNA sequences to be expressed or introduced into the same cell by cotransformation. The neomycin phosphotransferase (neo) gene is an example of a selectable marker gene. Additional elements may also be needed for optimal synthesis of mRNA. These elements may include signal sequences, splice signals, as well as transcriptional promoters, enhancers, and termination signals. Examples of expression vectors compatible with eukaryotic cells include pSVL and pKSV-10 (Pharmacia), pBPV-1, pML2d (International Biotechnologies), pTDT1 (ATCC 31255) and other eukaryotic expression vectors.

The recombinant expression vectors may carry sequences that regulate replication of the vector in host cells (e.g., origins of replication) and selectable marker genes. It will be appreciated by those skilled in the art that the design of the expression vector, including the selection of regulatory sequences may depend on such factors as the choice of the host cell to be transformed, the level of expression of protein desired, etc. Frequently used regulatory sequences for mammalian host cell expression include viral elements that direct high levels of protein expression in mammalian cells, such as promoters and enhancers derived from retroviral LTRs, cytomegalovirus (CMV) (such as the CMV promoter/enhancer), Simian Virus 40 (SV40) (such as the SV40 promoter/enhancer), adenovirus, (e.g., the adenovirus major late promoter (AdmP)), polyoma and strong mammalian promoters such as native immunoglobulin and actin promoters. For further description of viral regulatory elements, and sequences thereof, see e.g., Stinski, U.S. Pat. No. 5,168,062; Bell, U.S. Pat. No. 4,510,245; and Schaffner, U.S. Pat. No. 4,968,615, which are incorporated herein in their entireties.

### Genetically Modified Leukocytes

Various embodiments provide genetically modified leukocytes that have a resistance to prostanoids, where the resistance is reversible or can be overcome in a subject receiving the modified leukocytes. The genetically modified leukocytes contain an expression vector of a gene that confers resistance to prostanoids. The genetically modified leukocytes with resistance to prostanoids provide beneficial results to patients treated with prostanoids, or where prostanoids within the body reduce the activity of immune cells. Exemplary beneficial results include, or are characterized by, improved leukocyte survival and/or activity in the presence of prostanoids compared to unmodified, native leukocytes.

In various embodiments, at least 5%, 6%, 7%, 8%, 9%, 10%, 15%, 20% or 25% of a population of genetically modified cells are genetically modified leukocytes containing genes conferring resistance to prostanoids. In various embodiments, about 5%, 6%, 7%, 8%, 9%, 10%, 15%, 20% or 25% of a population of genetically modified cells are genetically modified leukocytes containing genes conferring resistance to prostanoids, or which contain with modifications to the prostacyclin receptor (PTGIR) gene that reduce or abolish PTGIR function. In various embodiments, up to 5%, 6%, 7%, 8%, 9%, 10%, 15%, 20% or 25% of a population of genetically modified cells are genetically modified leukocytes containing genes or alterations that confer resistance to prostanoids.

### Ablation or Silencing of PTGIR

Applicants discovered that ablating or silencing the expression of the prostacyclin receptor (PTGIR) in mouse or human CD8+ T cells as a means of overcoming CD8+ T cell dysfunction upon chronic exposure to virus or cancer. Downregulation of PTGIR partially restores functionality in adoptively transferred mouse CD8+ T cells when mice were challenged with a chronic strain of lymphocytic choriomeningitis virus (LCMV clone 13). Also, PTGIR silencing in adoptively transferred CD8+ T cells were found to reduce tumor volumes in mice challenged with syngeneic cancers including colorectal carcinoma (MC38-OVA), and melanoma (B16-OVA). The existing state-of-the-art therapy in mouse models involves adoptive transfer of CD8+ T cells without additional genetic aberrations other than genetically engineering cells to be antigen specific (i.e. target cancer cells expressing the OVA peptide in the mouse studies or antigens on human cancers such as, but not limited to, CD19, BCMA (B-Cell Maturation Antigen) or GPR5D (G-protein-coupled receptor, class C, group 5, member D)).

### Methods for PTGIR Modification in Immune Cells

In certain embodiments, the disclosure contemplates modifying immune cells such that the PTGIR gene is not able to produce a functioning PTGIR protein. Cells may be collected and obtained by isolation from a subject's or patient's peripheral blood and optionally purified by fluorescent activated cells sorting e.g., mixing cells with fluorescent antibodies or other fluorescent agents (molecular beacons) and separating the cells by flow cytometry based fluorescent sorting. Another option for cells sorting is to provide magnetic particles that are conjugated to specific binding agents, such as antibodies against a particular antigen on a target cells surface. After mixing with a sample, the antibody bound cells are put through a purification column containing a matrix composed of ferromagnetic spheres. When placed on a magnetic separator, the spheres amplify the magnetic field. The unlabeled cells pass through while the magnetically labeled cells are retained within the column. The flow-through can be collected as the unlabeled cells fraction. After a short washing step, the column is removed from the separator, and the magnetically labeled cells are eluted from the column.

CD3 is expressed on T cells as it is associated with the T cells receptor (TCR). The majority of TCR are made up of alpha beta chains (alpha beta T-cells). Alpha beta T-cells typically become double-positive intermediates (CD4+CD8+) which mature into single-positive (CD4+CD8−) T helper cells or (CD4−CD8+) cytotoxic T cells. T helper cells interact with antigen presenting dendritic cells and B cells. Upon activation with cognate antigen by dendritic cells, antigen-specific CD4 T cells can differentiate to become various types of effector CD4 T cells with specific roles in promoting immune responses. Mature gamma delta T cells are CD4− CD8− double-negative. T cells may be isolated and separated from a human sample (blood or PBMCs or bone marrow) based on the expression of alpha beta T cells receptor (TCR), gamma delta T cells receptor, CD2, CD3, CD4, CD8, CD4 and CD8, K1.1, CD4 and CD25 and other combinations based on positive or negative selection.

In certain embodiments, the immune cells are CD8+, CD4+, alpha beta T cells, delta gamma T cells, natural killer cells and/or double-negative alpha beta T cells. Wilhelm et al., report infusion of gamma delta T cells. J Transl Med. 2014; 12:45. Peripheral blood mononuclear cells (leukapheresis product) were depleted of CD4 and CD8 T-cells using anti-CD4 and anti-CD8 antibodies conjugated to paramagnetic particles. The procedure provides purified gamma delta T cells, NK cells, and double-negative alpha beta T cells.

### Strategies for Modifying the PTGIR

The modification of a PTGIR may be in the form of deleting, inserting, or altering the PTGIR gene or the nucleotide sequence that codes for the PTGIR. For example, a deletion or insertion of one nucleotide in an mRNA coding region would cause the codons for the rest of the sequences to be incorrect. Altering or changing a nucleotide may in result in a stop codon or an amino acid change. Desirable alterations are those that are amino acids that participate in the active cites of the enzyme or in known binding domains.

RNA interference (RNAi) or post-transcriptional gene silencing (PTGS) is a process by which expression of a specific gene is suppressed by small, double-stranded RNAs after transcription of the gene into mRNA but before translation of this message into protein. It can be triggered by short RNA molecules that regulate gene expression by binding to mRNA. This can occur as a natural process that silences genes, but it can also be applied experimentally with synthetic methods. RNA interference or post-transcriptional gene silencing can be performed by exogenous introduction of an interfering RNA or a trigger RNA. An interfering RNA can be small interfering RNAs (siRNA). An interfering RNA can also be a short hairpin RNA (shRNA). An interfering RNA can also be endogenous miRNAs that bind a target mRNA leading to translational suppression.

RNAi can be induced using synthetic small interfering RNA (siRNA) duplexes or using shRNA expressed from either a plasmid vector or from genomic DNA following lentiviral-mediated integration. Generally, siRNA is a synthetic, double-stranded RNA molecule, while shRNA is an RNA sequence that folds into a stem-loop structure. Typically, shRNA is processed by DICER before being incorporated into the RNA-induced silencing complex, while siRNA is not. While siRNA delivers the siRNA duplex directly to the cytosol, shRNAs are capable of DNA integration and consist of two complementary 19-22 bp RNA sequences linked by a short loop of 4-11 nt similar to the hairpin found in naturally occurring miRNA. The proper selection of a target sequence for a given gene of interest remains one of the most critical components of successful gene knockdown regardless of the RNAi methodology. See, e.g., Moore C B, Guthrie E H, Huang M T, Taxman D J. Short hairpin RNA (shRNA): design, delivery, and assessment of gene knockdown. Methods Mol Biol. 2010; 629:141-58. doi: 10.1007/978-1-60761-657-3_10. PMID: 20387148; PMCID: PMC3679364.

The siRNA is unwound during RISC assembly and the single-stranded RNA hybridizes with mRNA target. Gene silencing is a result of nucleolytic degradation of the targeted mRNA by the RNase H enzyme Argonaute (Slicer). If the siRNA/mRNA duplex contains mismatches the mRNA is not cleaved. Rather, gene silencing is a result of translational inhibition.

In some embodiments, vector-based short hairpin RNA (shRNA) can be used as the RNAi. shRNAs can be delivered to cells by lipid transfection of plasmid vectors or transduction by lentiviral particles. The shRNA sequence is encoded in the delivered sequence, and expression of the shRNA in the nucleus is driven either by an RNA Pol III promoter (including U6 or H1) or an RNA Pol II promoter (such as CMV).

The shRNA is cleaved by the endogenous dicer enzyme to generate the desired siRNA duplexes, which in turn associate with RNAi silencing complex (RISC). When shRNA is delivered using lentiviral vectors, the sequence encoding the shRNA is integrated into the genome and the knockdown effect is passed on to daughter cells, allowing for continued gene silencing.

Non-homologous end joining (NHEJ) is a pathway for double-strand break (DSB) repair in mammalian cells. As NHEJ is not dependent on the cells cycle, DSBs can be repaired via NHEJ in quiescent cells. In certain embodiments, genome editing entails genetic modification via the induction of a double-strand break (DSB) in a specific genomic target sequence, followed by the generation of desired modifications during subsequent DNA break repair. Modifications include gene disruption (the targeted induction of minor insertions and deletions), ‘gene correction’ (the introduction of discrete base substitutions specified by a homologous donor DNA construct) and targeted gene addition (the transfer of entire transgenes into a native genomic locus).

Strategies for modifying the PTGIR gene such that may include use of zinc-finger nucleases (ZFNs), transcription activator-like endonucleases (TALENs), and CRISPR-associated Cas9 endonucleases. ZEN have a Fokl cleavage domain. Repeating zinc fingers are on both sides of the Fokl. The zinc fingers specifically bind target nucleotide sequences. Combinatorial techniques can be used to produce zinc fingers that bind any nucleotide sequence. Fok I cleave the bases that separate the zinc fingers.

Transcription activator-like effector nucleases (TALENs) also contain a Fokl nuclease domain fused to a DNA-binding domain. This DNA-binding domain is composed of highly conserved repeats derived from proteins that are secreted in bacteria. See Reyon et al. FLASH assembly of TALENs for high-throughput genome editing. Nature Biotech. 30, 460-465 (2012).

Clustered regularly interspaced short palindromic repeats (CRISPR)-associated Cas9 (CRISPR/Cas9) systems may be engineered into vectors that function in human cells. The Cas9. crRNA complex introduces a double-strand break at a specific site in DNA containing a sequence complementary to crRNA. DNA cleavage is executed by the Cas9 protein. The RuvC and FINH domains generate nicks on opposite DNA strands. The Cas9-crRNA complex functions as an RNA-guided (gRNA) endonuclease with RNA-directed target sequence recognition and protein-mediated DNA cleavage. The gRNA can be altered in order to direct a DNA double-strand break (DSB) (from Cas9 endonuclease expressed in the cells) at a desired genomic location. Protospacer-adjacent motifs (PAMs) are short sequences that are typically required for Cas9 and the gRNA sequence to template the cleavage. Streptococcus pyogenes Cas9 can target sites flanked by S-NGG sequence.

### Chimeric Antigen Receptors

Antigen-specific CD8 T cells play an important role in controlling chronic infections or cancer but progressively lose their functions during prolonged antigen exposure. Such exhaustion of T cells limits the ability of the immune system to fully clear chronic infections or eradicate tumors. Recent approaches to restore CD8 T cells immune responses, such as engineering functional T cells with tumor-specific chimeric antigen receptors (CAR) improve the lifespan of certain patients with cancer.

CD19-CAR.S are indicated to be therapeutically effective at controlling lymphocytic leukemia. However, the modified T cells progressively lose their potency as they experience prolonged exposure to antigen. Thus, there is a need to extend the potency of CD19-CARs by blocking T cells exhaustion. Thus, in order to address T cells exhaustion, PTGIR from FACS purified donor T cells may be deleted in cells engineered to express the chimeric antigen receptor.

The CAR is typically made up of an antigen-binding polypeptide sequence conjugated to one or more intracellular T-cell signaling domains. A preferred CAR molecule contains the antigen binding domain, an intracellular T cells signaling domain, e.g., the immunoreceptor tyrosine-based activation motif of the CD3-zeta chain, and optionally additional signaling domains within the CAR construct, such as a CD28, CD137 (4-1BB) costimulatory signaling domains. For preparation, T cells may be transduced with lentiviral vector encoding a tumor associated chimeric antigen receptor. The antigen-binding portion may be a single-chain variable fragment derived from a monoclonal antibody that binds the tumor associated antigen or receptors consisting of heavy and light antibody chains fused to a T cells signaling molecule capable of activating the T cells response. Peripheral blood mononuclear cells (PBMCs) may be isolated by leukapheresis. T cells can be enriched by mononuclear cells elutriation and expanded by addition of anti-CD3/CD28 coated paramagnetic beads for activation of T cells. A lentiviral vector encoding the tumor associated chimeric antigen receptor may be added at the time of cells activation. Cells may be expanded, harvested and cryopreserved in infusible medium sometime after the subject has had an allogeneic stem-cell transplantation.

Suitable tumor associated antigens for targeting includes considerations of the target antigens based on tumor expression on normal tissues. It is preferred to pick optimal target tumor antigens that have no or limited expression on non-cancerous cells. Protein phage display libraries can be generated for testing based on the known sequences of the heavy chain antibodies that bind to antigens.

In certain embodiments, the PTGIR deficient T cells are engineered to express a chimeric antigen receptor (CAR) specific to an antigen associated with a cancer or chronic pathogen PTGIR deficient CAR-CD8 T cells may be adoptively transferred into the patient. Adoptive transfer T cells therapy of PTGIR deficient CD8 T cells may also be used in combination with antibodies to CD80/CTLA4 blockade, small molecule checkpoint inhibitors, interleukins, e.g., IL-2 (aldesleukin).

In certain embodiments, the PTGIR deficient T cells are engineered to express a chimeric antigen receptor (CAR) specific to a neoantigen, a class of tumor associated antigens is formed by peptides that are entirely absent from the normal human genome. For human tumors without a viral etiology, neo-epitopes are created by tumor-specific DNA alterations that result in the formation of novel protein sequences. For virus-associated tumors, such as cervical cancer and a subset of head and neck cancers, epitopes derived from viral open reading frames also contribute to the pool of neoantigens.

A large fraction of the mutations in human tumors is not shared between patients at meaningful frequencies and may therefore be considered patient-specific. Because of this, technologies to interrogate T cells reactivity against putative mutation-derived neoantigens need to be based on the genome of an individual tumor. With the development of deep-sequencing technologies, it has become feasible to identify the mutations present within the protein-encoding part of the genome (the exome) of an individual tumor and thereby predict potential neoantigens.

### Enhanced Immune Response Against Cancer

In certain embodiments, the modified PTGIR cells disclosed herein may be used in adoptive T cells therapies to enhance immune responses against cancer. In certain embodiments, this disclosure relates to methods of treating cancer comprising a) collecting immune cells or CD8 T cells from a subject (e.g. a subject who has been diagnosed with cancer), b) isolating the collected immune cells or CD8 T cells; c) modifying a PTGIR gene in the isolated immune cells or CD8 T cells such that the PTGIR gene does not expresses a protein or a non-functional protein is expressed providing an immune cells or CD8 T cells with an non-functioning PTGIR gene; d) administering or implanting an effective amount of the modified isolated immune cells or CD8 T cells into the subject (or a patient diagnosed with cancer).

In certain embodiments, the disclosure contemplates that the modified PTGIR T cells also express a chimeric antigen receptor (CAR) specific to a tumor associated antigen or neoantigen. In certain embodiments, the tumor associated antigen is selected from CD5, CD 19, CD20, CD30, CD33, CD47, CD52, CD152 (CTLA-4), CD340 (ErbB-2), GD2, TPBG, CA-125, CEA, MAGEA1, MAGE A3, MARTI, GP100, MUC1, WT1, TAG-72, HPVE6, HPVE7, BING-4, SAP-1, immature laminin receptor, vascular endothelial growth factor (VEGF-A) or epidermal growth factor receptor (ErbB-1). In certain embodiments, the tumor associated antigen is selected from CD20, CD20, CD30, CD33, CD52, EpCAM, epithelial cells adhesion molecule, gpA33, glycoprotein A33, Mucins, TAG-72, tumor-associated glycoprotein 72, Folate-binding protein, VEGF, vascular endothelial growth factor, integrin αvβ3, integrin α5β1, FAP, fibroblast activation protein, CEA, carcinoembryonic antigen, tenascin, Ley, Lewis Y antigen, CAIX, carbonic anhydrase IX, epidermal growth factor receptor (EGFR; also known as ERBB 1), ERBB2 (also known as HER2), ERBB3, MET (also known as HGFR), insulin-like growth factor 1 receptor (IGF1R), ephrin receptor A3 (EPHA3), tumor necrosis factor (T F)-related apoptosis-inducing ligand receptor 1 (TRAILR1; also known as TNFRSF10A), TRAILR2 (also known as T FRSF10B) and receptor activator of nuclear factor-KB ligand (RANKL; also known as T FSF11) and fragments thereof.

In certain embodiments, the T-cells specific to a tumor antigen can be removed from a tumor sample (TLs) or filtered from blood. Subsequent activation and culturing is performed outside the body (ex vivo) and then they are transfused into the patient. Activation may be accomplished by exposing the T cells to tumor antigens. In certain embodiments, the cancer is selected from multiple myeloma, hepatic cancer, pancreatic cancer, colon cancer, liver cancer, ovarian cancer, breast cancer, gastric cancer, lung cancer, melanoma, skin cancer, ovarian cancer, prostate cancer, head cancer, neck cancer, renal cancer, throat cancer, leukemia, and lymphoma.

In certain embodiments, the method further comprising administering in combination with an anticancer agent selected from gefitinib, erlotinib, docetaxel, cis-platin, 5-fluorouracil, gemcitabine, tegafur, raltitrexed, methotrexate, cytosine arabinoside, hydroxyurea, adriamycin, bleomycin, doxorubicin, daunomycin, epirubicin, idarubicin, mitomycin-C, dactinomycin and mithramycin, vincristine, vinblastine, vindesine, vinorelbine, taxol, taxotere, etoposide, teniposide, amsacrine, topotecan, camptothecin, bortezomib, anagrelide, tamoxifen, toremifene, raloxifene, droloxifene, iodoxyfene, fulvestrant, bicalutamide, flutamide, nilutamide, cyproterone, goserelin, leuprorelin, buserelin, megestrol, anastrozole, letrozole, vorazole, exemestane, finasteride, marimastat, trastuzumab, cetuximab, dasatinib, imatinib, bevacizumab, combretastatin, thalidomide, pomalidomide, and/or lenalidomide or combinations thereof.

In certain embodiments, chemotherapy aimed at depletion of T lymphocytes may be administered about a week before infusion of the CAR T cells.

In certain embodiments, the cancer is a hematological cancer, e.g., leukemia, multiple myeloma, or a lymphoma and administering the combination before or after autologous or allogeneic hematopoietic stem-cell transplantation.

### Coexpression System/Therapy

Genetically modified leukocytes generally contain a gene that confers prostanoid resistance of the invention, and in various embodiments, also co-expresses a transgene encoding an additional therapeutic effect. In some embodiments, the modified leukocytes of the present invention also co-expresses a transgene encoding for a recombinant T-cell receptor (TCR) gene or a chimeric T cell antigen receptor. In one aspect, (1) the polynucleotide chain conferring prostanoid resistance and (2) the polynucleotide chain encoding a recombinant TCR or a chimeric T cell antigen receptor, are linked by a nucleic acid sequence that encodes a cleavable linker. In another aspect, (1) the polynucleotide chain conferring prostanoid resistance and (2) the polynucleotide chain encoding a recombinant TCR or a chimeric T cell antigen receptor, are driven by independent promoters. In another aspect, the polynucleotides (1) and (2) may be linked by internal ribosome entry sequence (IRES). In yet another aspect, the polynucleotides (1) and (2) are present on, for example, two different vectors, which may be simultaneously or sequentially transfected or transduced. In another aspect, a combination gene may be used wherein polynucleotides (1) and (2) flank a polynucleotide sequence that encodes a cleavable polypeptide linker such that expression of the combination gene results in a single long polypeptide that is then cleaved into two polypeptides by cleavage of the polypeptide linker.

### Priming/Stimulation of the Leukocytes

In various embodiments, the genetically modified leukocytes of the present invention are also primed or stimulated with an antigen to confer an additional therapeutic effect. In some embodiments, the leukocytes are transfected and thereafter stimulated/primed with the antigen; in other embodiments, the leukocytes are first stimulated/primed with the antigen and then transfected with the gene that confers prostanoid resistance; and in yet other embodiments, the leukocytes are concurrently stimulated/primed with the antigen and transfected with the gene that confers prostanoid resistance.

### Pharmaceutical Composition and Dosage

A pharmaceutical composition is also provided including a population of genetically modified leukocytes and a pharmaceutically acceptable carrier or diluent. To facilitate administration, genetically modified leukocytes according to the invention can be made into a pharmaceutical composition or made into an implant appropriate for administration in vivo, with appropriate carriers or diluents, which further can be pharmaceutically acceptable. The means of making such a composition or an implant have been described, for instance, Remington's Pharmaceutical Sciences, 16th Ed., Mack, ed. (1980).

In some aspects, the genetically modified leukocytes can be formulated into a preparation in semisolid (e.g., encapsulated in hydrogel) or in liquid form, such as a capsule, solution, injection, inhalant, or aerosol, in the usual ways for their respective route of administration. Means known in the art can be utilized to prevent or minimize release and absorption of the composition until it reaches the target tissue or organ, or to ensure timed-release of the composition. Desirably, however, a pharmaceutically acceptable form is employed that does not ineffectuate the cells' resistance to prostanoids. Thus, genetically modified leukocytes, including T cells, can be made into a pharmaceutical composition containing a balanced salt solution, for example, Hanks' balanced salt solution, or normal saline.

Various embodiments provide the genetically modified leukocytes, or a pharmaceutical composition thereof, of the present invention can be provided in unit dosage form wherein each dosage unit, e.g., an injection, contains a predetermined amount of the population of genetically modified leukocytes or the composition, alone or in appropriate combination with other active agents. The term unit dosage form refers to physically discrete units suitable as unitary dosages for human and animal subjects, each unit containing a predetermined quantity of the genetically modified leukocytes of the present invention, alone or in combination with other active agents, calculated in an amount sufficient to produce the desired effect, in association with a pharmaceutically acceptable diluent, carrier, or vehicle, where appropriate. The specifications for the novel unit dosage forms of the present invention depend on the particular pharmacodynamic properties associated with the population of genetically modified leukocytes, or its pharmaceutical composition, in the particular subject.

For example, a single dosage contains about 1×104, 5×104, 1×105, 5×105, 1×106, 5×106, 1×107, or 5×107 of genetically modified leukocytes that contain a gene conferring resistance to prostanoids per kilogram of patient bodyweight. One or more dosage units may be administered to a subject depending on the condition of the subject and the therapeutic effects of the treatment. Multiple dosages may be administered at weekly, monthly or yearly intervals, where appropriate.

### Combination Therapy

In various embodiments, genetically modified leukocytes containing a gene that confers resistance to prostanoids is used or administered to a subject, in combination with glucocorticoid, nonsteroidal anti-inflammatory drugs, anti-infectives, or chemotherapeutics. The combination of therapies is used to treat, reduce the severity or likelihood, or slow the progression of auto-immune diseases or disorders, inflammatory disorders, infectious diseases or cancers.

In some aspects, genetically modified leukocytes exhibiting resistance of glucocorticoid, or a population thereof, are administered prior to glucocorticoid, nonsteroidal anti-inflammatory drugs, anti-infectives, or chemotherapeutics. In some aspects, genetically modified leukocytes exhibiting resistance of glucocorticoid, or a population thereof, are administered concurrently with glucocorticoid, nonsteroidal anti-inflammatory drugs, anti-infectives, or chemotherapeutics. In other aspects, modified leukocytes exhibiting resistance of glucocorticoid, or a population thereof, and glucocorticoid, nonsteroidal anti-inflammatory drugs, anti-infectives, or chemotherapeutics, are administered repeatedly as needed.

Exemplary glucocorticoids that may be administered concurrently or sequentially with the genetically modified leukocytes include, but are not limited to, cortisol, cortisone, prednisone, prednisolone, methylprednisolone, dexamethasone, betamethasone, triamcinolone, fludrocortisone acetate, and deoxycorticosterone acetate.

Exemplary nonsteroidal anti-inflammatory drugs (NSAIDs) that may be administered concurrently or sequentially with the genetically modified leukocytes include, but are not limited to, aspirin, celecoxib, diclofenac, diflunisal, etodolac, ibuprofen, indomethacin, ketoprofen, ketorolac, nabumetone, naproxen, oxaprozin, piroxicam, salsalate, sulindac, and tolmetin.

Exemplary anti-infectives that may be administered concurrently or sequentially with the genetically modified leukocytes include, but are not limited to, antibiotics, antifungals, anthelmintics, antimalarials, antiprotozoals, antituberculosis agents, and antivirals.

Exemplary chemotherapeutics that may be administered concurrently or sequentially with the genetically modified leukocytes include, but are not limited to, alkylating agents (e.g., mechlorethamine, cyclophosphamide, melphalan, chlorambucil, ifosfamide and busulfan; N-Nitroso-N-methylurea (MNU), carmustine (BCNU), lomustine (CCNU) and semustine (MeCCNU), fotemustine and streptozotocin; dacarbazine, mitozolomide and temozolomide; thiotepa, mytomycin and diaziquone); antimetabolites (e.g., anti-folates, fluoropyrimidines, deoxynucleoside analogues and thiopurines); anti-microtubule agents (e.g., vinorelbine, vindesine, and vinflunine); topoisomerase inhibitors (e.g., irinotecan, topotecan, camptothecin, etoposide, doxorubicin, mitoxantrone, teniposide, novobiocin, merbarone, and aclarubicin); and cytotoxic antibiotics (e.g., doxorubicin, daunorubicin, leukocyteepirubicin, idarubicin, pirarubicin, aclarubicin, mitoxantrone, actinomycin, bleomycin, and mitomycin).

### Methods of Using the Cells Providing Prostanoid Resistance

In various embodiments, a method of treating, reducing the severity or likelihood, or slowing the progression of a disease in a mammal includes administering a pharmaceutical composition that contains genetically modified leukocytes that express a gene that confers resistance to prostanoids. In some aspects, the method of treating, reducing the severity or likelihood, or slowing the progression of a disease in a mammal includes administering a pharmaceutical composition that contains genetically modified leukocytes that express a gene that confers resistance to prostanoids.

Exemplary diseases or disorder to be treated or reduced severity or likelihood of by administering genetically modified leukocytes include neoplasms (e.g. cancers, leukemias and lymphomas), infections caused by microorganisms or viruses (e.g. human cytomegalovirus (CMV), BK-virus, adenovirus), auto-immune disorders (e.g. type I diabetes, systemic lupus erythromatosus (SLE), Hashimoto's thyroiditis), acquired or congenital immune-deficiencies and hematopoietic stem cell transplantations. Examples of autoimmune diseases that may be treated with the genetically modified leukocytes of the claimed subject matter include, but are not limited to, acute idiopathic thrombocytopenia purpura, chronic idiopathic thrombocytopenic purpura, dermatomyositis, Sydenham's chorea, myasthenia gravis, systemic lupus erythematosus, lupus nephritis, rheumatic fever, polyglandular syndromes, bullous pemphigoid, diabetes mellitus, Henoch-Schonlein purpura, post-streptococcalnephritis, erythema nodosum, Takayasu's arteritis, Addison's disease, rheumatoid arthritis, multiple sclerosis, sarcoidosis, ulcerative colitis, erythema multiforme, IgA nephropathy, polyarteritis nodosa, ankylosing spondylitis, Goodpasture's syndrome, thromboangitisubiterans, Sjogren's syndrome, primary biliary cirrhosis, Hashimoto's thyroiditis, thyrotoxicosis, scleroderma, chronic active hepatitis, polymyositis/dermatomyositis, polychondritis, pamphigus vulgaris, Wegener's granulomatosis, membranous nephropathy, amyotrophic lateral sclerosis, tabes dorsalis, giant cell arteritis/polymyalgia, peraiciousanemia, rapidly progressive glomerulonephritis, psoriasis eosinophilic esophagitis and fibrosing alveolitis. Exemplary diseases or disorder to be treated or reduced severity or likelihood of by administering genetically modified leukocytes along with treating or pre-treating the patient with glucocorticoids include side-effects of delivering immune cells to patients, cytokine release syndrome, severe inflammatory syndrome, vascular leak, hypotension, pulmonary edema, neurotoxicity, cerebral edema, hemophagocytic lymphohistiocytosis or macrophage activation syndrome and mast cell activation syndrome.

### Transplantation in Patients

In some embodiments, a subject suitable for receiving the genetically modified leukocytes of the present invention is a patient receiving transplantation. For example, in allogeneic kidney transplantation, patients may be medicated with immunosuppressive drugs such as cyclosporine, sirolimus, mycophenolate mofetil or tacrolimus in combination with a steroid to prevent rejection of the graft by components of the immune system. Immunocompromised patients are at risk of developing uncontrolled viral infections such as BK virus (BKV) and human cytomegalovirus (CMV). For example, recipients of bone marrow (BMT) or renal transplants, may experience severe renal and urological complications from BKV. There is currently no effective therapeutic for BKV, and the only treatment available in solid organ transplantation is a reduction of immunosuppressive treatment—including glucocorticoids—with the concomitant risk of graft rejection.

### Clinical Application

In general, if adoptively transferred gene modified leukocytes are resistant to prostanoids while cells in the recipient remained responsive to the immunosuppressive effects of prostanoids, the administration of prostanoids to such a patient would reduce the function of such endogenous cells while preserving the activity of the adoptively transferred gene modified prostanoid resistant leukocytes.

Anti-viral T cells can be generated from peripheral blood obtained from a bone marrow donor against CMV, EBV or adenovirus. To make these anti-viral T cells, bone marrow donor blood leukocytes are exposed to viral antigens in vitro using recombinant adenoviral vectors modified to express CMV proteins, and EBV lymphoblastoid cell lines. During this process, the cell population can be transduced or transected with vectors containing a gene that confers prostanoid resistance as defined herein, or subject to genetic modifications that reduce or abolish the function of the PTGIR gene, e.g a CRISPR-mediated mutation that destroys functional expression of PTGIR gene product at one or more PTGIR alleles.

Prostanoid resistant anti-viral T cells can be infused into a patient. A preferred embodiment is where at least ten percent of genetically modified leukocytes express a gene that confers resistance to prostanoids.

Example implementations and embodiments will now be described more fully with reference to the accompanying drawings. Example implementations and embodiments are provided so that this disclosure will be thorough and will fully convey the scope of the disclosure to those of ordinary skill in the art. Specific details are set forth such as examples of specific components, materials, compositions, and methods, to provide a thorough understanding of implementations and embodiments of the present disclosure. It will be apparent to those of ordinary skill in the art that specific details need not be employed, that example implementations and embodiments may be embodied in many different forms, and that the specific details and the example implementations and embodiments should not be construed to limit the scope of the disclosure.

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3rd ed., Revised, J. Wiley & Sons (New York, N.Y. 2006); March, Advanced Organic Chemistry Reactions, Mechanisms and Structure 7th ed., J. Wiley & Sons (New York, N.Y. 2013); and Sambrook and Russel, Molecular Cloning A Laboratory Manual 4th ed., Cold Spring Harbor Laboratory Press (Cold Spring Harbor, N.Y. 2012), provide one skilled in the art with a general guide to many of the terms used in the present application.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1—Cloning and Transfection and Laboratory Work

Genes were obtained from commercial vendors (e.g Origene, Integrated DNA Technologies, Life Technologies, Thermo Fischer, Invitrogen) and cloned into vectors obtained from the same commercial vendors. Such vendors also supply transfection reagents, cell lines, tissue culture media, and reagents to perform analyses of cells and enzyme activity. Chemicals obtained from vendors such as Sigma Aldrich and TCI. Genetic analyses and flow cytometry analyses were obtained core laboratories at Van Andel Research Institute and external commercial vendors as needed.

The conferring of prostanoids resistance was assessed by methods including: (i) survival in the presence of glucocorticoids, measured by methods including but not limited to: cell counts, cell viability, MTT assay, apoptosis assays (tube or plate-based, imaging, flow cytometry), (ii) using techniques to measure reduction in immune activity, (iii) in vivo studies of virus or tumor growth.

### Construction of Retrovectors Containing Short Hairpin RNA

Short hairpin sequences were generated according to the protocols in Dow, L., Premsrirut, P., Zuber, J. et al. A pipeline for the generation of shRNA transgenic mice. Nat Protoc 7, 374-393 (2012).//doi.org/10.1038/nprot.2011.446.

The short hairpin control “FF” shRNA (S′-3′): AGCTCCCGTGAATTGGAATCCTAGTGAAGCCACAGATGTAGGATTCCAATTCAGCGG GAGCC (SEQ ID NO: 1) and shPTGIR shRNA (5′-3′): AGCACGAGAGGATGAAGTTTACTAGTGAAGCCACAGATGTAGTAAACTTCATCCTCT CGTGCC (SEQ ID NO: 2) were cloned by PCR using Xho1 forward primers and EcoR1 reverse primers and ligated into EcoR1/Xho1 digested pLMPd-Ametrine vector. The PCR product was run on 1% agarose gel then the cleaned up using the Qiaquick gel extraction kit (Qiagen, cat #28104). The cleaned PCR product, and the pLMPD-Ametrine plasmid were then digested overnight at 37 deg C. using EcoR1 and Xho1. The vector was then treated with calf intestinal phosphatase for 1H at 37 deg C. (NEB, cat #M02920). Products from the digest were then cleaned up (not run on gel) using the QIAquick gel extraction kit (Qiagen, cat #28704). The ligation product was then transformed into DH5alpha cells, according to standard procedures, then grown on a LB-AMP (100 μg/mL) plate. Single colonies were selected, expanded in LB-AMP broth, then sent for sequencing to validate the presence and orientation of the insert. After transformation into DH5alpha bacteria, cells were plated on Luria Broth agar with ampicillin (100 u/ml). Single colonies were selected and expanded in LB-AMP broth. Clones were DNA sequenced to validate the presence and orientation of desired insert.

### Production of Retroviral Vectors

Retroviral vectors were produced in accordance with standard protocols. 293T cells were cultured on 10 cm plates. Retro vector plasmid DNA and helper plasmid was mixed with Lipofectamine 2000 and OptiMEM (Thermo Fisher), and exposed to the 293T cells, followed by incubation for 6 hours to overnight. Supernatants containing retroviral particles were collected. Harvested supernatant was filtered through a 0.45 μm filter or centrifuged at 1400 rpm for 5 min to remove any detached 293T cells. Supernatants were stored at 4° C. or one-time frozen at −80° C. before use. Supernatants were concentrated using a LentiX concentrator (Takara Bio) in accordance with manufacturer's instructions.

### T Cell Transduction by Retroviral Vectors.

T cells were activated for 24 hours by exposure to a tissue culture plate coated with anti-CD3 and anti-CD28 antibodies. For spin transduction, activated T-cells were exposed to LentiX concentrated retrovirus in the presence of 8 ug/ml Polybrene and 1e5 U/ml Interleukin 2. The culture vessel was centrifuged at 1181 g at 30 degree angle for 90 minutes.

Cloning shRNAs Against Human PTGIR Sequences.

Human PTGIR gene (Gene ID: 5739, Transcript NM_000960.4) is targeted by sequences at various regions include Coding sequence (CDS), 3′ untranslated region (3UTR)). shRNA against PTGIR include those constructed using the following sequences.

| | Target | Match | Forward Oligo | Reverse Oligo |
|---|---|---|---|---|
| CloneID | Sequence | Region | Sequences | Sequences |
| TRCN0000014180 | GCCGTGGGA | CDS | CCGGGCCGTGGGAACGTC | AATTCAAAAAGCCGTG |
| | ACGTCGTCC | | GTCCAAACTCGAGTTTGG | GGAACGTCGTCCAAAC |
| | AAA (SEQ ID | | ACGACGTTOCCACGGCTT | TCGAGTTTGGACGACG |
| | NO: 3) | | TTTG | TTCCCACGGC |
| | | | (SEQ ID NO: 4) | (SEQ ID NO: 28) |
| TRCN0000357987 | AGGGACAGA [symbol] | 3UTR | CCGGAGGGACAGAGGAA | AATTCAAAAAAGGGAC |
| | AACGTTTAT | | ACGTTTATCTCGAGATAA | AGAGGAAACGTTTATC |
| | (SEQ ID NO: 5) | | ACGTTTCCTCTGTCCCTTT | TCGAGATAAACGTTTC |
| | | | TTTG | CTCTGTCCCT |
| | | | (SEQ ID NO: 6) | (SEQ ID NO: 29) |
| TRCN0000358060 | CTGCTCCCT | CDS | CCGGCTGCTCCCTCTGCT | AATTCAAAAACTGCTC |
| | CTGCTGACA | | GACATTTCTCGAGAAATG | CCTCTGCTGACATTTCT |
| | TTT | | TCAGCAGAGGGAGCAGTT | CGAGAAATGTCAGCAG |
| | (SEQ ID NO: 7) | | TTTG | AGGGAGCAG |
| | | | (SEQ ID NO: 8) | (SEQ ID NO: 30) |
| TRCN0000358057 | CGTCCATGCT [symbol] | CDS | CCGGCGTCCATGCTCATC | AATTCAAAAACGTCCA |
| | CCTCTTTG | | CTCTTTGCTCGAGCAAAG | TGCTCATCCTCTTTGCT |
| | (SEQ ID NO: 9) | | AGGATGAGCATGGACGTT | CGAGCAAAGAGGATG |
| | | | TTTG | AGCATGGACG |
| | | | (SEQ ID NO: 10) | (SEQ ID NO: 31) |
| TRCN0000358058 | CTCCAATCT | 3UTR | CCGGCTCCAATCTGCGTC | AATTCAAAAACTCCAA |
| | GCGTCCACT | | CACTTAGCTCGAGCTAAG | TCTGCGTCCACTTAGCT |
| | TAG | | TGGACGCAGATTGGAGTT | CGAGCTAAGTGGACGC |
| | (SEQ ID NO: 11) | | TTTG | AGATTGGAG |
| | | | (SEQ ID NO: 12) | (SEQ ID NO: 32) |
| TRCN0000014178 | CCCATCCAT | 3UTR | CCGGCCCATCCATCTCAT | AATTCAAAAACCCATC |
| | CTCATTGTC | | TGTCTAACTCGAGTTAGA | CATCTCATTGTCTAACT |
| | TAA | | CAATGAGATGGATGGGTT | CGAGTTAGACAATGAG |
| | (SEQ ID NO: | | TTTG | ATGGATGGG |
| | 13) | | (SEQ ID NO: 14) | (SEQ ID NO: 33) |
| TRCN0000014179 | CTTCCGCTT | CDS | CCGGCTTCCGCTTCTACG | AATTCAAAAACTTCCG |
| | CTACGCCT | | CCTTCAACTCGAGTTGAA | CTTCTACGCCTTCAACT |
| | TCAA | | GGCGTAGAAGCGGAAGT | CGAGTTGAAGGCGTAG |
| | (SEQ ID NO: | | TTTTG | AAGCGGAAG |
| | 15) | | (SEQ ID NO: 16) | (SEQ ID NO:34) |
| TRCN0000014182 | CTACGCCTTC [symbol] | CDS | CCGGCTACGCCTTCTGCG | AATTCAAAAACTACGC |
| | GTCC | | TCCTCTTCTCGAGAAGAG | CTTCTGCGTCCTCTTCT |
| | TCTT | | GACGCAGAAGGCGTAGTT | CGAGAAGAGGACGCA |
| | (SEQ ID NO: 17 [symbol] | | TTTG | GAAGGCGTAG |
| | | | (SEQ ID NO: 18) | (SEQ ID NO: 35) |
| TRCN0000014181 | CCTCAGCCTC [symbol] | CDS | CCGGCCTCAGCCTCTGCC | AATTCAAAAACCTCAG |
| | CGC ATGTA | | GCATGTACTCGAGTACAT | CCTCTGCCGCATGTAC |
| | (SEQ ID NO: 19 [symbol] | | GCGGCAGAGGCTGAGGTT | TCGAGTACATGCGGCA |
| | | | TTTG | GAGGCTGAGG |
| | | | (SEQ ID NO: 20) | (SEQ ID NO: 36) |

[symbol]

indicates data missing or illegible when filed

### Designs for Human PTGIR Knockout

The designs for human PTGIR (NM_000960.4) knockout via CRISR Sequences were generated using CRISPick (Broad Institute). For example, these sequences include:

a. Sequence 1 (5′-3′):
(SEQ ID NO: 21)
GCGCGCATAGGCCACGAACA
b. Sequence 2 (5′-3′):
(SEQ ID NO: 22)
GAAGGCGAAGGCATCGCACA
c. Sequence 3 (5′-3′):
(SEQ ID NO: 23)
GCTGCGCGTAGAGGTAGGGG

### Cell Preparation

Cell cultures were incubated at 37° C. in an atmosphere of 5% CO2 in air.

### Example 2—Silencing PTGIR

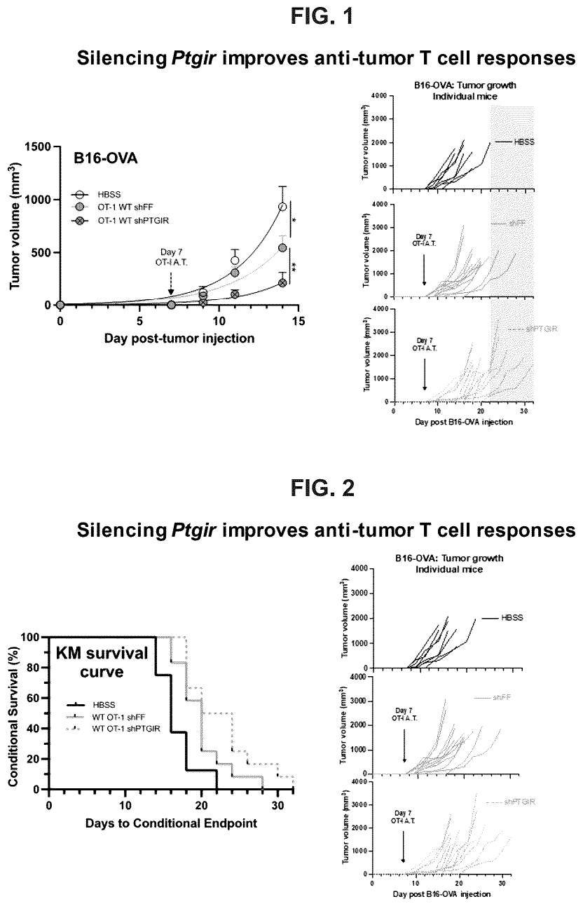

Referring to FIG. 1, this indicates a normal mouse CD8+ treated with a construct expressing: control short hairpin RNA (shFF) or short hairpin RNA against mouse PTGIR gene. OT-1 is an anti-OVA T cell receptor that recognizes ovalbumin epitope (OVA) antigen variant E1 (EIINFEKL) restricted by H2-Kb. Mouse T cells that constitutively express OT-1 (“OT-1 cells”) were transduced with a construct expressing short hairpin RNA against mouse Ptgir gene (“shPTGIR”) or control short hairpin RNA (“shFF”; against firefly luciferase).

At day 0, mice were inoculated with SE5 B16-OVA melanoma cells (these cells constitutively express the OVA protein and present OVA epitopes on cell surface MHC). On Day 7 mice received (i.v.) 1E6 adoptively transferred gene modified OT-1 cells or Hank's buffered saline solution (HBSS).

At day 0, mice inoculated with B16-OVA melanoma. On Day 7 mice received adoptively transferred, gene modified CD8+ cells on Day 7 or saline (HBSS). As seen by the “X” circle plot, mice that received the OT-1 cells transduced with shPTGIR had reduced tumor growth, suggesting the shPTGIR OT-1 cells mounted a stronger immune response against the B16-OVA melanoma.

### Example 3—Improved Anti-Tumor T Cell Responses

Referring to FIG. 2, this example was performed in the same way as Example 2. As seen by the “X” circle plot, mice that received the transferred T cells with the shPTGIR had increased survival.

### Example 4—Enhanced Antigen-Specific T Cell Expansion in Chronic Infection

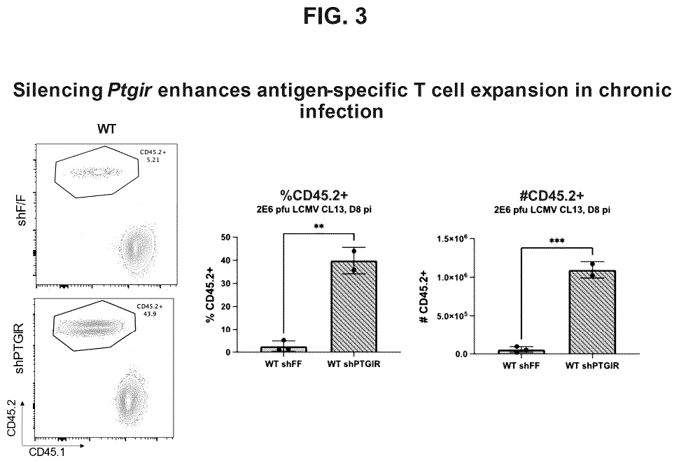

Referring to FIG. 3, this example shows data from a mouse model of chronic infection, LCMV (Lymphocytic choriomeningitis). This demonstrates that transduction of shPTGIR into effector T-cells results in a robust immune response and T-cells that have reduced phenotypic markers of exhaustion.

The model has two components: 1. A source of adoptive effector cells which is a transgenic mouse stain the Tera Knockout/Transgenic LCMV P14 T Cell Receptor strain. This strain is homozygous for a transgene that encodes a T cell receptor that is specific for a peptide (P14) from the lymphocytic choriomeningitis virus (LCMV's protein “gp” residues 33-41) presented by MHC lass I molecule H-2 Db. Almost all the peripheral T cells are CD8+ and express the transgenic TCR. Further, in our work, the P14 transgene was on background that has the CD45.2 allele, allowing for identification of transferred cells in CD45.1 congenic hosts.

CD45.1 mice were dosed with 2E6 plaque forming units (pfu) of LCMV (Lymphocytic choriomeningitis) clone 13. The animals received 1E5 CD45.2+P14 cells which were transduced with shRNA against Ptgir (“shPTGIR”) or control short hairpin RNA against firefly luciferase (“shFF”). The animals received a combination of equal numbers of anti-LCMV CD8+ T-cell from congenic mice carrying the CD45.1 or CD45.2 surface markers, respectively. Prior to infusion, the cells from CD45.2 donors were transfected with shRNA against PTGIR or control “FF or F/F”.

At day 8 post infection, cell populations were analyzed. There was a highly significant expansion of adoptively transferred cells (CD45.2+) which had been treated with anti-Ptgir shRNA compared to the control shFF.

### Example 5—Silencing PTGIR Enhances Antigen-Specific T Cell Expansion

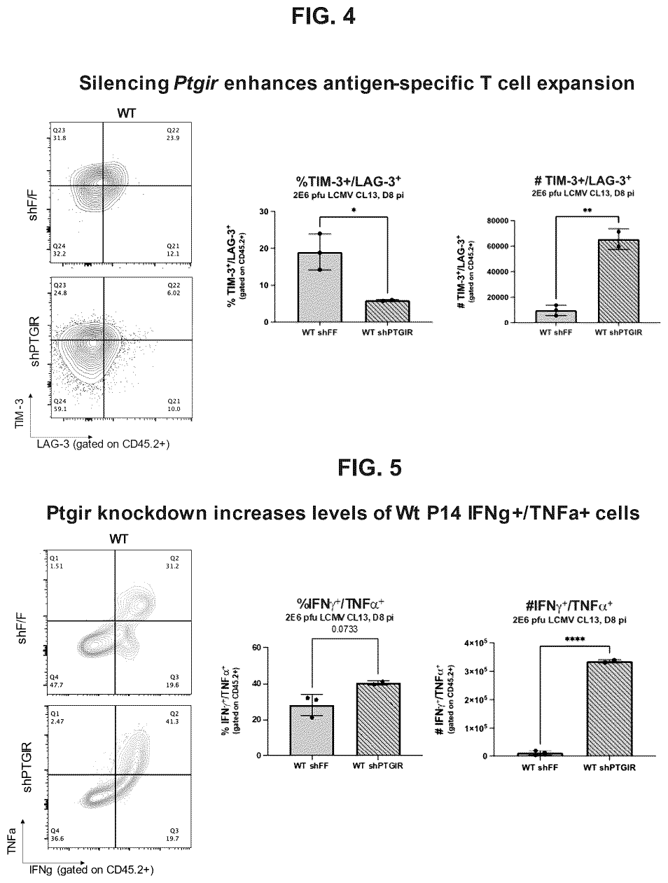

Referring to FIG. 4, this example was performed in the same way as Example 3.

Tim-3 expression on T-cells during an immune response to LCMV Lis correlated with cells that are short-term and do not provide lasting immunity). The figure shows that in T-cells carrying the CD45.2 surface marker, T-cells transduced with shPtgir had higher absolute abundance (right bar charts) and lower percentage of Tim-3 (left bar chart).

LAG-3 has multiple roles in T cells during viral infections including maintaining CD8 cells in tolerogenic state and promoting exhaustion. As seen in the flow cytometry panel, the shPtgir cells have lower LAG-3 expression compared to controls (shFF), suggesting they are not displaying an exhausted phenotype.

The figure shows that T-cells carrying the CD45.2 surface marker, T-cells transduced with shPtgir had higher absolute abundance (right bar charts) and lower percentage of Tim-3 (left bar chart).

As seen in the flow cytometry panel, the shPTGIR cells have lower LAG-3 expression compared to controls (shFF), suggesting they are, on relative level (as percentage of its own population) not displaying an exhausted phenotype (left hand bar graph). The right hand bar graph shows that shPTGIR transduced cells are present in greater absolute numbers compared to P14 cells treated with shFF.

In combination, low Tim-3 and low LAG-3 are positive indications for strong and persisting anti-viral responses. Cells treated with shPTGIR display low Tim-3 and LAG-3 expression, suggesting lower exhaustion.

### Example 6—PTGIR Knockdown

Referring to FIG. 5, this example was performed in the same way as Example 5. This demonstrates the results from a flow cytometry of adoptively transferred P14 CD8+CD45.2+ T-cells recovered from host animals infected with LCMV clone 13. The Figure shows intracellular staining for interferon gamma (IFNγ) and tumor necrosis factor alpha (TNFα) after cells were stimulated ex vivo by LCMV peptides and Interleukin 2. This shows that these shPTGIR T-cells cells recovered from the animals are capable of responding to antigen stimulation, which is further evidence supporting their non-exhausted state (e.g. see prior slide TIM-3/LAG-3).

PTGIR knockdown was shown in increased levels of wild type cytokines including interferons (IFNs), and tumor necrosis factor (TNF) in cells. One of the first responses against viral infections is the production of anti-viral cytokines, including interferons (IFNs), and tumor necrosis factor (TNF). Once produced, cytokines act directly on both infected and uninfected cells, activate cellular constituents of the innate immune system, and promote T and B cell responses. The effects of these cytokines are pleiotropic and influenced by dose as well as the presence or absence of other cytokines. This demonstrates that targeting PTGIR could be an alternative means to override the cytoprotective effects of the NRF2 pathway and boost CD8+ T cell function.

### Example 7—Ptgir mRNA Expression Correlated with Elevated NRF2 Gene Structure

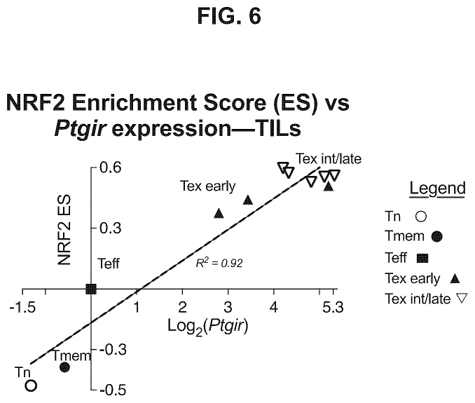

Referring to FIG. 6, Applicants have discovered a correlation between nuclear factor erythroid 2-related factor 2 (NRF2) activity and Ptgir expression in CD8 T cell subsets. In this example, raw sequences from RNA-seq of CD8+ T cells from three previously published studies (GEO accessions: GSE89307, GSE84820, and GSE86881) were downloaded and analysis was conducted as described in the methods section by Luda and Longo et al. (PMID: 37516105), with the following exceptions: Differential gene expression analyses of all Tex (early in black triangles, intermediate/late in white triangles) cells versus Teff cells were conducted on raw counts using DESeq2, with a covariate to adjust for batch, and Benjamini-Hochberg adjusted p-values to maintain a 5% false discovery rate.

Applicants founds that relative Ptgir mRNA expression (Log2 fold-change) versus NRF2 pathway enrichment scores (NFE2L2.V2) in CD8 T cells from Listeria monocytogenes infection indicated as naïve/Tn (in white circle), effector/Teff (in black square), and memory/Tmem (in black circle) or exhausted/Tex CD8 TIL isolated from early (<21 days) versus intermediate/late (>21 days) stage liver tumors (data from Philip et al. (PMID: 28514453)). Expression values were normalized to Teff cells. Linear regression indicates goodness of fit (R2=0.92).

### Example 8—Overexpression of PTGIR Linked to Tumor Growth

Applicants found that tumor targeting CD8+ T-cells that overexpress PTGIR were shown to poorly control tumor growth in a murine melanoma model. In this example, for retrovirus production corresponding to PTGIR overexpression in CD8 T cells, 293T cells were first co-transfected with pCL-Eco and either MIGR1 (GFP control vector, kindly provided by Warren Pear, #27490 Addgene), or MIGR1-PTGIR (custom PTGIR overexpressing vector VectorBuilder), using Lipofectamine 2000 transfection reagent and subsequent procedures as mentioned above, with the following exception: 72 h post-transduction GFP-positive cells were flow sorted, cultured for 48 h in T cell media, phenotypically validated for PTGIR overexpression, and adoptively transferred into tumor-bearing hosts.

For the targeted quantitation, parallel reaction monitoring (PRM) was performed on an Exploris 480 mass spectrometer coupled with the Vanquish Neo LC system (Thermo Fisher Scientific). A total of 2 μg of digested peptides were separated on a nano capillary column (20 cm×75 μm I.D., 365 μm O.D., 1.7 μm C18, CoAnn Technologies, Washington) at a flow rate of 300 nL/min. Mobile phase A was water with 0.1% formic acid, and mobile phase B was 20% water and 80% acetonitrile with 0.1% formic acid. The LC gradient was as follows: 1% B to 26% B over 51 minutes, 85% B over 5 minutes, and 98% B over 4 minutes, with a total gradient length of 60 minutes. Full MS spectra (m/z 375-1200) were collected at a resolution of 120,000 (FWHM), and MS2 spectra at 30,000 resolutions (FWHM). Standard AGC targets and automatic maximum injection times were used for both full and MS2 scans. A 32% HCD collision energy was used for MS2 fragmentation. All samples were analyzed using a multiplexed PRM method with a scheduled inclusion list containing the target precursor ions. Two unique peptides of PTGIR (SEQ ID No. 24: GFTQAIAPDS and SEQ ID No. 25: EMGDLLAFR) were used to measure the relative abundance of PTGIR, while two alpha-actinin4 peptides (SEQ ID No. 26: DDPVTNLNNAFEVAEK and SEQ ID No. 27: LVSIGAEEIVDGNAK) served as internal standards.

DIA data were processed in Spectronaut (version 18, Biognosys, Switzerland) using directDIA™ analysis. Data were searched against the Mus musculus reference proteome database (Uniprot, Taxon ID: 10090) with the manufacturer's default parameters. Briefly, trypsin/P was set as the digestion enzyme, allowing for two missed cleavages. Cysteine carbamidomethylation was set as a fixed modification, while methionine oxidation and protein N-terminus acetylation were set as variable modifications. Identification was performed using a 1% q-value cutoff at both the precursor and protein levels. Both peptide precursor and protein false discovery rates (FDR) were controlled at 1%. Ion chromatograms of fragment ions were used for quantification, with the area under the curve between the XIC peak boundaries calculated for each targeted ion. DDA raw files were utilized in Library Extension Runs to enhance proteome coverage to generate a hybrid library. All PRM data analysis and integration were performed using Skyline software. The transitions' intensity rank order and chromatographic elution were required to match those of a synthetic standard for each measured peptide. All proteomics sample preparation, LC-MS/MS and analysis was conducted by the VAI mass spectrometry core RRID:SCR_024903.

In this example, 8-12 week-old female C57BL/6 mice were injected with 5×10⁵ B16-OVA melanoma cells subcutaneously in the right abdominal flank. Once palpable tumors were present, tumor measurements were obtained every 2-3 days using a caliper. Mice were euthanized as they reached humane endpoints, which included a maximum tumor volume of 1500 mm3. For adoptive transfer experiments, on day 7 post inoculation, and when palpable tumors were present, 1×10⁶ OT-I T cells were adoptively transferred into tumor-bearing hosts.

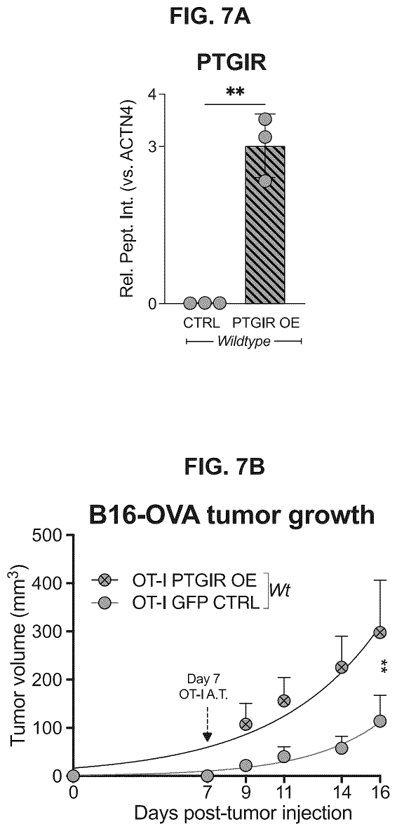

Referring to FIG. 7A, this shows that relative PTGIR protein expression (normalized to ACTN4) in WT OT-I T cells following transduction with empty vector (CTRL) or PTGIR-expressing (PTGIR OE) retroviral vector (mean+/−SEM, n=3). This data is represented using a two-tailed, unpaired Student's t-test to compare the means between two independent groups.

Referring to FIG. 7B, this shows B16-OVA melanoma tumor growth in mice that received adoptive transfer of control (CTRL) or PTGIR-expressing (PTGIR OE) WT OT-I cells at 7 dpti (mean+/−SEM, n=8). ** P<0.01. The control is indicated in gray circle. The PTGIR OE is indicated in the x-marked circles. The tumor growth curve statistical analysis between groups was conducted via mixed-effects model with exponential (Malthusian) growth non-linear, or third-order polynomial (cubic) fit.

### Example 9—Low PTGIR Expression Linked to Increase Survival in Human Cancer Patients

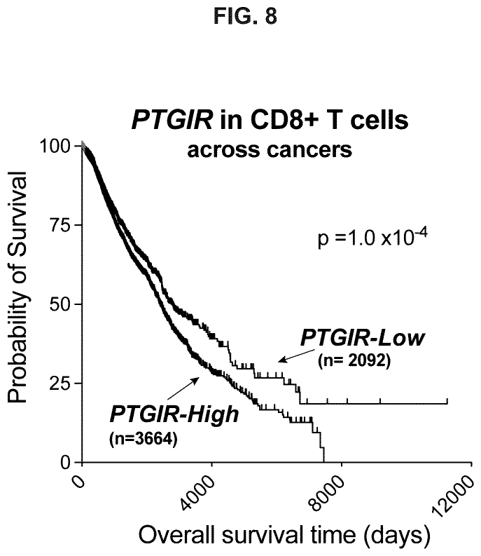

Referring to FIG. 8, Applicants have shown that human cancer patients bearing CD8+ T-cells with low PTGIR expression exhibited increased overall survival. In this example, Kaplan-Meier (KM) survival curves examining prostacyclin receptor (PTGIR) mRNA expression. Patients with low PTGIR expression (PTGIRLo) within CD8+T subsets of tumor cells have increased survival probability. For statistical analysis, a log-rank (Mantel-Cox) test with p values. (n= # of patients). Patient data was obtained from the UCSC XENA database. As shown in FIG. 8, for patients with high PTGIR expression (n=3664), that probability of survival curve exhibits a downward shift in which the 25% survival at less than 5,000 days. By contrast, for patients with low PTGIR expression (n=2092), the probability of survival curve shows a 25% survival at about 6,000-12,000 days.

### Construct/Vector Design

Due to common amino sequences in 2A sequences (e.g., C-terminal NPGP using the single letter amino acid code) alternate codon usage may be required to encode 2A regions to avoid interference with the function of a gene construct, for example, by homologous or complementary sequences impeding cloning, preventing proper vector generation and function or generating secondary structure within the RNA transcript or with a delivered nucleic acid such as an RNA that may directly encode a protein.

Incorporation of a cell surface tag into a poly-cistronic gene construct permits the detection of transduction of the gene construct as well as isolation of cells using isolation techniques such as flow cytometry or bead-based purification techniques, where such isolation techniques are generally known in the field. Detection of expressed proteins, including cell surface tags, is afforded through reagents with antigen or epitope-specific affinity such as antibodies or aptamers. The detection of the cell surface tag gives a strong indication that cleavage has occurred and that gene products are present in the cell. Intracellular antigens can be detected following permeabilization of a cell and may be conducted instead of, or in concert with, detection of antigens expressed on the cell surface. Binding of epitope-specific affinity reagent to its target antigen or epitope can be detected by standard methods including direct or indirect means that are well known in the field including flow cytometry, mass cytometry, ELISA, enzyme assays and fluorescent microscopy.

Examples of direct detection include, but are not limited to, creating a modified antibody which contains structures that can be detected such as fluorophore dyes and quantum dots, stable isotopes, nucleic acid tags, nuclear magnetic resonance (NMR) tags, enzymes including ribozymes, biotin, and radioisotope. Indirect means include using reagents that bind to the primary antibody. Examples of indirect detection include, but are not limited to, detecting binding of a primary antibody using a secondary reagent where such secondary reagent is labeled and where such labels can be chosen from the list of detection materials including but not limited to fluorophore dyes and quantum dots, stable isotopes, nucleic acid tags, nuclear magnetic resonance (NMR) tags, enzymes including ribozymes, biotin and radioisotope. Examples of secondary reagents include but is not limited to avidin, antisera, monoclonal antibodies, aptamers, Fc region binding proteins, anti-idiotype antibodies and peptides that bind to grooves, clefts or pockets in antibody structures or aptamers.

Various embodiments of the invention are described above. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.).

What is claimed is:

1. A population of leukocytes comprising CD8 T-cells that have been genetically modified to reduce or silence expression in the CD8 T-cells of a functional prostaglandin 12 receptor (PTGIR) gene or of a functional PTGIR protein.

2. The population of leukocytes of claim 1 wherein the expression of PTGIR is disrupted by introducing an interfering RNA into the CD8 T-cells.

3. The population of leukocytes of claim 1 wherein the interfering RNA is a short hairpin RNA (shRNA).

4. The population of leukocytes of claim 1 wherein the interfering RNA is small interfering RNA (siRNA).

5. The population of leukocytes of claim 1 wherein the expression of PTGIR is disrupted with a CRISPR system.

6. The population of leukocytes of claim 1 wherein the expression of PTGIR is disrupted by knocking down the PTGIR.

7. The population of leukocytes of claim 1 wherein the expression of PTGIR is disrupted by knocking out the PTGIR.

8. The population of leukocytes of claim 1 wherein the expression of PTGIR is disrupted by the prevention of ribosomes from synthesizing the PTGIR.

9. The population of leukocytes of claim 1 wherein the expression of PTGIR is disrupted by marking the mRNA that codes for PTGIR for destruction.

10. The population of leukocytes of claim 1 wherein the disrupted expression of PTGIR improves anti-tumor T-cell responses.

11. The population of leukocytes of claim 1 wherein the disrupted expression of PTGIR PTGIR enhances antigen-specific T-cell expansion in chronic infection.

12. The population of leukocytes of claim 1 wherein the disrupted expression of PTGIR increases levels of Wt P14 IFNγ+/TNFα+ cells.

13. The population of leukocytes of claim 1 wherein the CD8 T-cells are regulatory T-cells (Tregs).

14. The population of leukocytes of claim 1 wherein the CD8 T-cells are suppressor T-cells.

15. The population of leukocytes of claim 1 wherein the CD8 T-cells are tumor infiltrating lymphocytes.

16. A method of disrupting expression of prostaglandin 12 receptor (PTGIR) in a CD8 T-cell comprising:
introducing an interfering RNA into a CD8 T-cell;
allowing the interfering RNA to bind an mRNA that codes for PTGIR;
reducing or silencing the expression of PTGIR;
reducing prostacyclin binding; and
enhancing CD8 T cell activity

17. The method of claim 16 wherein the interfering RNA is a short hairpin RNA (shRNA).

18. The method of claim 16 wherein the interfering RNA is small interfering RNA (siRNA).

19. The method of claim 16 wherein the expression of PTGIR is disrupted with a CRISPR system.

20. The method of claim 16 wherein the expression of PTGIR is disrupted by knocking down the PTGIR.

21. The method of claim 16 wherein the expression of PTGIR is disrupted by knocking out the PTGIR.

22. The method of claim 16 wherein the expression of PTGIR is disrupted by the prevention of ribosomes from synthesizing the PTGIR.

23. The method of claim 16 wherein the expression of PTGIR is disrupted by marking the mRNA that codes for PTGIR for destruction.

24. The method of claim 16 wherein reducing or silencing the expression of PTGIR improves anti-tumor T-cell responses.

25. The method of claim 16 wherein reducing or silencing the expression of PTGIR enhances antigen-specific T-cell expansion in chronic infection.

26. The method of claim 16 wherein reducing or silencing the expression of PTGIR increases levels of Wt P14 IFNγ+/TNFα+ cells.

27. A method of treating cancer comprising:
collecting CD8 T-cells from a subject;
isolating the collected CD8 T-cells;
genetically modifying the isolated CD8 T-cells such that the expression of a prostaglandin 12 receptor (PTGIR) in the CD8 T-cells is disrupted by reducing the PTGIR or preventing the PTGIR gene in the CD8 T-cells from expressing a functional protein; and
administering a pharmaceutically effective amount of the modified isolated CD8 T-cells into a patient.

28. The method of claim 27 wherein the subject from who the CD8 T-cells were collected and the patient are the same.

29. The method of claim 27 wherein the subject from who the CD8 T-cells were collected and the patient are different.


SEQUENCE LISTING (36 sequences)

SEQ ID NO: 1 - RNA, 62 nt, synthetic construct
  source 1..62: mol_type = other RNA
agctcccgtgaattggaatcctagtgaagccacagatgtaggattccaattcagcgggagcc

SEQ ID NO: 2 - RNA, 63 nt, synthetic construct
  source 1..63: mol_type = other RNA
agcacgagaggatgaagtttactagtgaagccacagatgtagtaaacttcatcctctcgtgcc

SEQ ID NO: 3 - RNA, 21 nt, synthetic construct
  source 1..21: mol_type = other RNA
gccgtgggaacgtcgtccaaa

SEQ ID NO: 4 - RNA, 58 nt, synthetic construct
  source 1..58: mol_type = other RNA
ccgggccgtgggaacgtcgtccaaactcgagtttggacgacgttcccacggctttttg

SEQ ID NO: 5 - RNA, 21 nt, synthetic construct
  source 1..21: mol_type = other RNA
agggacagaggaaacgtttat

SEQ ID NO: 6 - RNA, 58 nt, synthetic construct
  source 1..58: mol_type = other RNA
ccggagggacagaggaaacgtttatctcgagataaacgtttcctctgtcccttttttg

SEQ ID NO: 7 - RNA, 21 nt, synthetic construct
  source 1..21: mol_type = other RNA
ctgctccctctgctgacattt

SEQ ID NO: 8 - RNA, 58 nt, synthetic construct
  source 1..58: mol_type = other RNA
ccggctgctccctctgctgacatttctcgagaaatgtcagcagagggagcagtttttg

SEQ ID NO: 9 - RNA, 21 nt, synthetic construct
  source 1..21: mol_type = other RNA
cgtccatgctcatcctctttg

SEQ ID NO: 10 - RNA, 58 nt, synthetic construct
  source 1..58: mol_type = other RNA
ccggcgtccatgctcatcctctttgctcgagcaaagaggatgagcatggacgtttttg

SEQ ID NO: 11 - RNA, 21 nt, synthetic construct
  source 1..21: mol_type = other RNA
ctccaatctgcgtccacttag

SEQ ID NO: 12 - RNA, 58 nt, synthetic construct
  source 1..58: mol_type = other RNA
ccggctccaatctgcgtccacttagctcgagctaagtggacgcagattggagtttttg

SEQ ID NO: 13 - RNA, 21 nt, synthetic construct
  source 1..21: mol_type = other RNA
cccatccatctcattgtctaa

SEQ ID NO: 14 - RNA, 58 nt, synthetic construct
  source 1..58: mol_type = other RNA
ccggcccatccatctcattgtctaactcgagttagacaatgagatggatgggtttttg

SEQ ID NO: 15 - RNA, 21 nt, synthetic construct
  source 1..21: mol_type = other RNA
cttccgcttctacgccttcaa

SEQ ID NO: 16 - RNA, 58 nt, synthetic construct
  source 1..58: mol_type = other RNA
ccggcttccgcttctacgccttcaactcgagttgaaggcgtagaagcggaagtttttg

SEQ ID NO: 17 - RNA, 21 nt, synthetic construct
  source 1..21: mol_type = other RNA
ctacgccttctgcgtcctctt

SEQ ID NO: 18 - RNA, 58 nt, synthetic construct
  source 1..58: mol_type = other RNA
ccggctacgccttctgcgtcctcttctcgagaagaggacgcagaaggcgtagtttttg

SEQ ID NO: 19 - RNA, 21 nt, synthetic construct
  source 1..21: mol_type = other RNA
cctcagcctctgccgcatgta

SEQ ID NO: 20 - RNA, 58 nt, synthetic construct
  source 1..58: mol_type = other RNA
ccggcctcagcctctgccgcatgtactcgagtacatgcggcagaggctgaggtttttg

SEQ ID NO: 21 - RNA, 20 nt, synthetic construct
  source 1..20: mol_type = other RNA
gcgcgcataggccacgaaca

SEQ ID NO: 22 - RNA, 20 nt, synthetic construct
  source 1..20: mol_type = other RNA
gaaggcgaaggcatcgcaca

SEQ ID NO: 23 - RNA, 20 nt, synthetic construct
  source 1..20: mol_type = other RNA
gctgcgcgtagaggtagggg

SEQ ID NO: 24 - protein, 10 aa, synthetic construct
GFTQAIAPDS

SEQ ID NO: 25 - protein, 9 aa, synthetic construct
EMGDLLAFR

SEQ ID NO: 26 - protein, 16 aa, synthetic construct
DDPVTNLNNAFEVAEK

SEQ ID NO: 27 - protein, 15 aa, synthetic construct
LVSIGAEEIVDGNAK

SEQ ID NO: 28 - RNA, 58 nt, synthetic construct
  source 1..58: mol_type = other RNA
aattcaaaaagccgtgggaacgtcgtccaaactcgagtttggacgacgttcccacggc

SEQ ID NO: 29 - RNA, 58 nt, synthetic construct
  source 1..58: mol_type = other RNA
aattcaaaaaagggacagaggaaacgtttatctcgagataaacgtttcctctgtccct

SEQ ID NO: 30 - RNA, 58 nt, synthetic construct
  source 1..58: mol_type = other RNA
aattcaaaaactgctccctctgctgacatttctcgagaaatgtcagcagagggagcag

SEQ ID NO: 31 - RNA, 58 nt, synthetic construct
  source 1..58: mol_type = other RNA
aattcaaaaacgtccatgctcatcctctttgctcgagcaaagaggatgagcatggacg

SEQ ID NO: 32 - RNA, 58 nt, synthetic construct
  source 1..58: mol_type = other RNA
aattcaaaaactccaatctgcgtccacttagctcgagctaagtggacgcagattggag

SEQ ID NO: 33 - RNA, 58 nt, synthetic construct
  source 1..58: mol_type = other RNA
aattcaaaaacccatccatctcattgtctaactcgagttagacaatgagatggatggg

SEQ ID NO: 34 - RNA, 58 nt, synthetic construct
  source 1..58: mol_type = other RNA
aattcaaaaacttccgcttctacgccttcaactcgagttgaaggcgtagaagcggaag

SEQ ID NO: 35 - RNA, 58 nt, synthetic construct
  source 1..58: mol_type = other RNA
aattcaaaaactacgccttctgcgtcctcttctcgagaagaggacgcagaaggcgtag

SEQ ID NO: 36 - RNA, 58 nt, synthetic construct
  source 1..58: mol_type = other RNA
aattcaaaaacctcagcctctgccgcatgtactcgagtacatgcggcagaggctgagg